(19) United States
(12) Patent Application Publication
(10) Pub. No.: US 2026/0216406 A1
(43) Pub. Date: Jul. 30, 2026

(54) DEVICES AND METHODS OF A MEDICAMENT-PRODUCING IMPLANTABLE MATRIX

(71) Applicant: Humacyte Global, Inc., Durham, NC (US)

(72) Inventors: Mehmet H. KURAL, Cary, NC (US); Kaleb NAEGELI, Durham, NC (US); Hong QIAN, Durham, NC (US); Yuling LI, Durham, NC (US); Laura E. NIKLASON, Greenwich, CT (US)

(21) Appl. No.: 19/146,748

(22) PCT Filed: Jan. 11, 2024
(86) PCT No.: PCT/US2024/011240

§ 371 Date: Jul. 9, 2025

Related U.S. Application Data
(60) Provisional application No. 63/438,444, filed on Jan. 11, 2023.

(51) Int. Cl. A61L 27/38; A61L 27/22; A61L 27/44; A61L 27/54

(52) U.S. Cl. CPC A61L 27/3834; A61L 27/222; A61L 27/44; A61L 27/54

(57) ABSTRACT

Described herein are variations of a medicament-producing implantable matrix for the treatment of chronic diseases. For example, an implantable matrix may include a cell-coated a human acellular vessel-based vascular graft. The cells may be engineered to produce medicaments capable of treating a chronic disease. A cell-seeded matrix may be implanted adjacent to a patient's blood vessel such that the medicaments produced by the cells are easily released into the patient's blood stream. Additionally, or alternatively, a cell-seeded matrix may treat chronic disease at an environment local to an implantation site. Also described herein are methods for making and use of such implantable matrices.

This application claims the benefit of U.S. Provisional Pat. App. No. 63/438,444, which was filed on Jan. 11, 2023, of which is incorporated by reference in its entirety.

### FIELD OF THE DISCLOSURE

The present disclosure relates to medicament-producing implantable matrices and methods of making thereof.

### BACKGROUND

Patients with chronic diseases such as cancer, multiple sclerosis, rheumatoid arthritis, hemophilia A (i.e., factor VIII (FVIII) deficiency or classic hemophilia), Chron's disease, ulcerative colitis, and ankylosing spondylitis are often dependent on pharmaceuticals for treatment. Current pharmaceutical drug technology is mostly based on infusion delivery of cytokines, hormones, antibodies, or recombinant proteins produced in cell, yeast, or bacterial culture. Typically, these pharmaceuticals are administered via infusion therapy to deliver treatment directly into the bloodstream, but this procedure can cause complications such as phlebitis and infection at the injection site. Additionally, intravenous therapy sessions can be time consuming-some are required once or twice a week for up to two hours- and patients can have lifelong dependencies on these treatments. Moreover, some of these repetitive procedures cause immense financial burden for the patients. Thus, there is a need for new and improved delivery of pharmaceutical agents to chronically diseased patients. Accordingly, the present disclosure provides devices and methods to produce therapeutic molecules (medicaments) via implantable matrices in patients' bodies.

### BRIEF SUMMARY

In some embodiments, a medicament-producing implantable matrix is provided which comprises a matrix comprising at least one population of cells.

In some embodiments, a medicament-producing implantable matrix is provided which comprises a frame comprising collagen and a matrix comprising at least one population of cells, where the matrix is at least one of attached to and within the frame.

In some embodiments, a method of forming a medicament-producing implantable matrix is provided and comprises combining at least one population of cells with a solubilized matrix, casting the combination into a frame; and polymerizing the solubilized matrix.

In some embodiments, an apparatus is provided which comprises a tubular support graft comprising a matrix and at least one population of cells, where the tubular support graft is configured to be concentrically positioned around a vascular graft.

In some embodiments, an apparatus is provided which comprises a tubular support graft, where the tubular support graft is configured to be concentrically positioned around a tubular vascular graft, and a matrix comprising at least one population of cells and disposed on an external surface of the tubular support graft.

In some embodiments, the present disclosure further relates to a method for forming a tubular support graft, comprising arranging a support mandrel within a tubular mold, a first end of the support mandrel and a first end of the tubular mold being fluidly sealed by a first end cap, injecting a mixture of hydrogel and a cell suspension within a volume formed between surfaces of the support mandrel and the tubular mold, fluidly sealing a second end of the support mandrel and a second end of the tubular mold via a second end cap, and removing the second end cap and the tubular sleeve after curing of the mixture to form the tubular support graft.

In some embodiments, a method for forming a cell-populated tubular support graft for a tubular vascular graft is provided and comprises mounting a tubular support graft to a support mandrel, rotating the support mandrel at a predetermined speed, depositing, via a 3-dimensional printer, droplets of a mixture of hydrogel and at least one population of cells onto an external surface of the support mandrel, and after curing, removing the cell-populated tubular support graft from the support mandrel.

Some variations of at least the above-noted embodiments may further include one and/or another, and in some variations, a plurality of, and in some variations (if not mutually exclusive), substantially all of, the following elements, features, functionality, structure, materials, steps, and/or clarifications, yielding yet further variations of embodiments of the disclosure:
the at least one population of cells produces one or more molecules selected from the group consisting of: Ocrelizumab, Natalizumab, Pembrolizumab, Infliximab, Vedolizumab, Dabrafenib, Lecanemab, or interferon beta-1a;
the at least one population of cells produces one or more molecules selected from the group consisting of: Factor VII, Factor VIII, Factor IX, Factor X, Von Willebrand factor, Protein C, human albumin, human Immune Globulin, testosterone, human Htt, or P42.
the at least one population of cells produces interleukin-10;
the at least one population of cells produces sirolimus or tacrolimus;
the at least one population of cells comprises mesenchymal stem cells;
the at least one population of cells comprises major histocompatibility complex class I and II knock-out mesenchymal stem cells;
the matrix comprises at least one selected from the group consisting of: a hydrogel and a biodegradable polymer;
the matrix comprises a hydrogel comprising thrombin and fibrinogen, where the thrombin can be at a concentration of between about 0.1 units/mL and about units mg/mL; or
the matrix comprises a hydrogel comprising thrombin and fibrinogen, where the fibrinogen can be at a concentration of between about 3 mg/mL and about 100 mg/mL;
the matrix comprises a Fas receptor activating molecule;
the Fas receptor activating molecule comprises a Fas ligand;
the matrix is implanted into at least one body region of a patient selected from the group consisting of: knees, arms, legs, hips, elbows, wrists, spine, stomach, blood vessels, and intestines;
the matrix can be formed in the desired implantation site by injecting the cells in a hydrogel glue solution; and
the matrix comprises at least one selected from the group consisting of: a hydrogel and a biodegradable polymer, where:
   the biodegradable polymer is one selected from the group consisting of: polyglycolic acid, polylactic acid, poly(lactic-co-glycolic) acid, poly(caprolactone); and/or
   the hydrogel is at least one of a naturally-derived hydrogel and a synthetic hydrogel;
the vascular graft can include one or more selected from the group consisting of: fibrin, collagen, alginate, gelatin, chitosan, dextran, hyaluronic acid, or PEG;
the vascular graft can include a combination of: PEG and chitosan, PEG and gelatin, PEG and hyaluronic acid, PAM and gelatin, or PVA and gelatin;
the tubular support graft can include one or more selected from the group consisting of: fibrin, collagen, alginate, gelatin, chitosan, dextran, hyaluronic acid, or PEG;
the tubular support graft can include a combination of: PEG and chitosan, PEG and gelatin, PEG and hyaluronic acid, PAM and gelatin, or PVA and gelatin;
the tubular support graft is formed by one of tubular molding, casting, planar tissue culture, electrospinning, and 3-dimensional printing;
a length of the tubular support graft is less than a length of the tubular vascular graft;
a length of the tubular support graft is greater than a length of the tubular vascular graft;
a length of the tubular vascular graft is between 1 cm and 100 cm;
forming a tubular support graft includes placing the tubular support graft on the outer surface of a vascular graft;
forming a tubular support graft includes implanting the vascular graft carrying the tubular support graft into an arm of a patient;
forming a tubular support graft includes implanting the vascular graft carrying the tubular support graft adjacent to a leg of a patient.

These and other objects, features, advantages of at least some of the embodiments of the disclosure will be even more clear with reference to the following drawings, a brief description of which immediately follows, as well as detailed description set out thereafter.

### BRIEF DESCRIPTION OF THE SEVERAL VIEWS OF THE DRAWINGS

FIG. 1 depicts an implantable matrix with a tissue substrate, hydrogel substrate, cells, and additives, according to some variations.

FIG. 2 depicts an implantable matrix with a tissue substrate, cells, and additives, according to some variations.

FIG. 3 depicts an implantable hydrogel matrix, cells, and additives, according to some variations.

FIG. 4 depicts an implantable hydrogel matrix, a support mesh, cells, and additives, according to some variations.

FIGS. 5A-5B depict an implantable matrix with a tissue frame and hydrogel interior.

FIG. 6 depicts an illustrative schematic of a method of preparing a tissue substrate for use in an implantable matrix.

FIG. 7 depicts an illustrative schematic of a variation of a method for attaching a tissue substrate to a hydrogel substrate for use as an implantable matrix.

FIG. 8 depicts an illustrative schematic of a variation of a method for 3-D printing a tubular cell-populated hydrogel (i.e., tubular support graft).

FIG. 9 depicts an illustrative schematic of a variation of a method for placing a cell-populated hydrogel tube (i.e., tubular support graft) onto a vascular graft, where the tubular support graft is comprises a hydrogel, cells and additives, according to some variations.

FIG. 10 depicts an illustrative schematic of a tubular cell-populated hydrogel (tubular support graft), according to some variations. The tubular support graft can be a cell-hydrogel mixture comprising cells within the hydrogel.

FIG. 11 depicts a tubular vascular graft with cell-populated coating (tubular support graft) anastomosed to a patient's artery and vein.

FIGS. 12A-12C depicts implantable matrix patches attached to implantation sites of interest include vertebrae, intestines, and joint linings.

FIG. 13 depicts an illustrative schematic of a variation of a method for producing a tubular cell-populated hydrogel (i.e., tubular support graft) by casting a hydrogel mixture into the space between the cylindrical mandrel and a tubular mold. Upon curing/polymerization of the hydrogel, the tubular support graft and the mandrel can be removed from the mold.

### DETAILED DESCRIPTION

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Non-limiting examples of various aspects and variations of the invention are described herein and illustrated in the accompanying drawings.

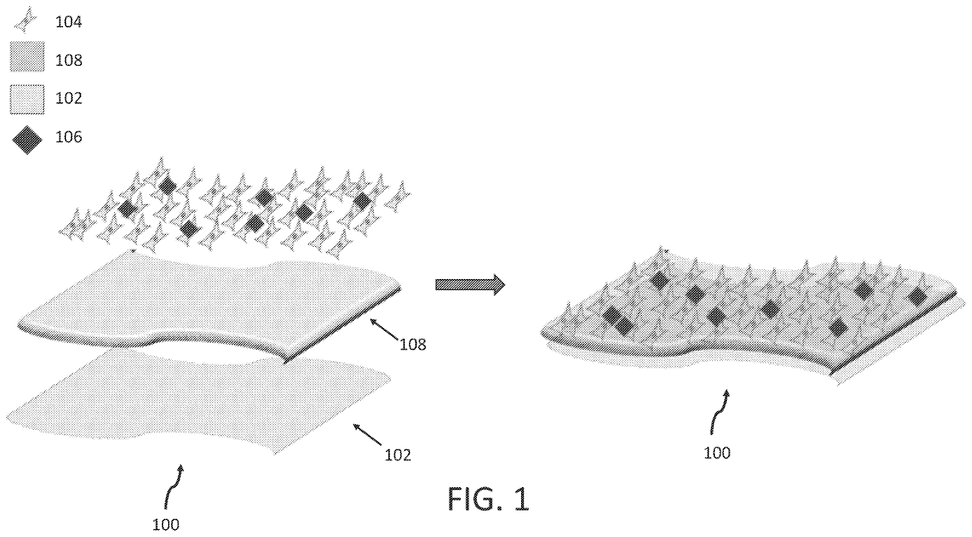

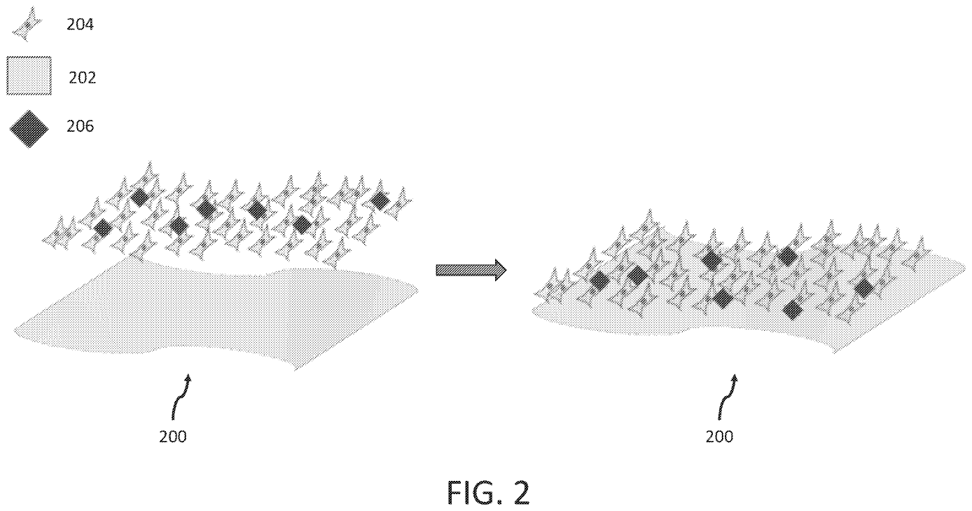

As generally described herein, a medicament-producing implantable matrix may include a matrix populated with medicament-secreting cells. The matrix may, for example, include a cell laden hydrogel attached to or coated onto a patch of decellularized tissue. A cell-populated hydrogel matrix may facilitate local delivery of medicaments via seeded cells, which may evade an immune response to the implantable matrix. The medicament-secreting cells may, for example, be engineered to produce medicaments (i.e., drugs, agents) to treat a particular chronic disease. The biovascular medicament-producing implantable matrix may be anastomosed to a patient's vein or artery such that the secreted agents are released into the patient's bloodstream. A decellularized tissue substrate of the implantable matrix may facilitate attachment (e.g., suturing, gluing) and anastomosis of the matrix to an implantation site. In some variations, the matrix can be formed as a coating on the outer surface of a vascular graft and can be anastomosed to the blood circulation via vascular surgery. For example, as shown in FIG. 1, implantable matrix 100 may include cells 104 in hydrogel 108 which is coated onto tissue patch 102. Cells may be mixed into a hydrogel precursor solution and may include more than one cell population. For example, in some variations, cells 104 may include medicament-producing cells and IL-10 expressing cells or immunosuppressant drug-producing cells to alleviate an immune reaction to implantable matrix 100. This co-delivery of cell populations may locally protect the medicament-producing cells from an immune response. As shown in FIG. 2, an implantable matrix 200 may alternatively include tissue patch 202 which may include cells 204 seeded directly onto its outer surface. This biovascular implantable matrix may facilitate systemic treatment of chronic diseases, as medicament-secreting cells 104 may be proximal to a patient's circulatory system when surface-seeded. Referring again to FIG. 1, in some variations, implantable matrix 100 may further include additives 106, which may be seeded into a hydrogel matrix. Additives may, for example, further alleviate an immune reaction to cells, or may generally support the physical properties of hydrogel 108 and/or tissue patch 102 or may generally support cell proliferation in vivo.

Alternatively, the medicament-producing cells described herein may be contained in an implantable pouch or similar device and delivered to an implantation site of interest.

The implantable matrices described herein have characteristics that improve numerous aspects of treatment of chronic diseases. For example, treatment via an implantable matrix may only require one procedure to implant the matrix, saving patients from complications, time, and money spent on ongoing pharmaceutical treatment. Further, as described in detail herein, the implantable matrix may provide additionally support to an implantation site and may provide local protection of medicament-producing cells.

Although the medicament-producing systems described herein may be described with reference to local or systemic treatment of chronic diseases (e.g., cancer, multiple sclerosis, rheumatoid arthritis, hemophilia, Chron's disease, ulcerative colitis, and ankylosing spondylitis, etc.), it should be understood that such systems may additionally or alternatively be configured to treat other illnesses or disorders such as genetic disorders.

Various aspects of example variations of the medicament-producing implantable matrix, and methods of making and use thereof, are described in further detail below. Section 1 discusses implantable matrix devices. Section 2 discusses cell secretion of medicaments and (optionally) additives for implantable matrices. Section 3 discusses fabrication of implantable matrices. Section 4 discusses implantation of matrices. Section 5 discusses examples relating to implantable matrix devices and methods described herein.

### SECTION 1: IMPLANTABLE MATRIX

As shown in FIG. 1, implantable matrix 100 may generally include tissue substrate 102 and hydrogel 108 to deliver medicament-producing cells 104 to an implantation site and support their growth and proliferation in vivo. In some variations, an implantable matrix may further include a hydrogel glue substrate and/or additives 106. As discussed in detail herein, in some variations, an implantable matrix may include either the tissue substrate 102 or the hydrogel 108.

In general, an implantable matrix may be populated with at least one cell. In some variations, the substrate may have a thickness between about 1 μm and about 5 cm, such as between about 1 μm and about 5 mm, between about 10 μm and about 1 mm, between about 50 μm and about 500 μm, or between about 100 μm and about 250 μm. In some variations, a surface area of a matrix substrate may be between about 1 μm² and about 100 cm², such as between about 10 μm² and about 10 cm², between about 100 μm² and about 1 cm², between about 1 mm² and about 500 mm², or between about 100 mm² and 250 mm². Figures referred to herein show the implantable matrix to be a rectangular patch or a tubular graft, but it should be understood that a matrix may be any suitable shape to assist in systemic delivery of medicaments, such as a circle, a triangle, an oval, an irregular shape, or a pouch configured to contain medicament-producing cells.

### Tissue Substrate

As shown in FIG. 2, implantable matrix 200 may include tissue substrate 202. In some variations, tissue substrate 202 may be used to deliver surface seeded cells 204 proximally to a patient's circulatory system such that the medicament(s) secreted by cells 204 are easily delivered into the bloodstream. In some variations, tissue substrate 202 may also include surface seeded additives 206. Tissue substrate 202 may be a patch of decellularized tissue and may be biodegradable and biocompatible such that it temporarily supports proliferation of seeded cells at an implantation site as it is enzymatically degraded by a patient's body. For example, a collagen-based matrix may be enzymatically degraded and mediated through natural means by proteins such as collagenase. Degradation processes of matrices described herein may be on the order of days, weeks, months, or years. In some variations, tissue substrate 202 may be an artificial acellular substrate or a decellularized tissue substrate. In some variations, blood vessel tissue, skin, or tendon may be decellularized to produce tissue substrate 202. For example, a regenerative vascular conduit may be decellularized to produce tissue substrate 202 (e.g., Human Acellular Vessel). In some variations, tissue substrate 202 may be collagenous. Collagen and other mammalian-derived protein-based polymers may provide effective matrices for cellular growth because they contain many cell-signaling domains present in the in vivo extracellular matrix. Additionally, the mechanical properties of collagen (toughness, firmness, inelasticity) can aid with implantation of an implantable matrix. Collagen substrates can be created through natural means without chemical modifications. Various methods have been developed to synthesize substrates with enhanced mechanical properties such as chemical crosslinking, UV or thermal crosslinking, and/or mixing with other polymeric agents. In some variations, an artificial acellular tissue substrate may be fabricated from, for example, fibrin, collagen, alginate, gelatin, chitosan, dextran, hyaluronic acid, PEG, PEG/Chitosan, PEG/Gelatin, PEG/hyaluronic acid, PAM/Gelatin, or PVA/Gelatin. Typically, cells 204 and/or additives 206 may be seeded on an outer surface of tissue substrate 202. Typically, a thickness of tissue substrate 202 may be between about 1 μm and about 5 cm, such as between about 10 μm and about 1 cm, between about 100 μm and about 5 mm, or between about 250 μm and about 1 mm. In some variations, a thickness of tissue substrate 202 is about equal to a thickness of a decellularized blood vessel such as HAV. For example, the thickness of tissue substrate 202 may be about 500 μm.

In variations where a tissue substrate is a rectangular patch, the tissue substrate may have side lengths between about 1 mm and about 10 cm, such as between about 1 cm and about 9 cm, between about 2 cm and about 8 cm, or between about 3 cm and about 7 cm. Side lengths of the tissue substrate may be the same or different. For example, the tissue substrate may have a width of about 2 cm and a length of about 2 cm, or a width of about 2 cm and a length of about 5 cm. In some variations, a width of the tissue substrate may be about equal to a circumference of a decellularized blood vessel such as HAV.

### Hydrogel Matrix

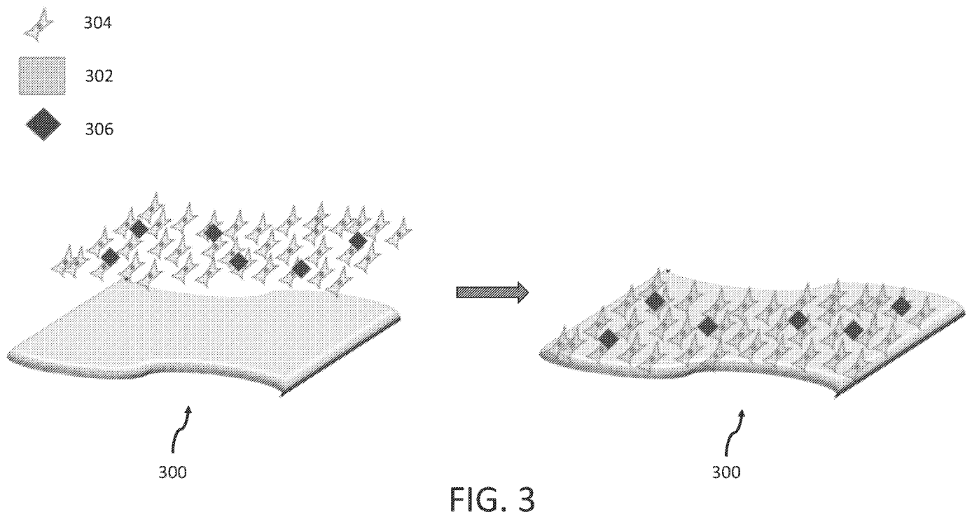

Referring to FIG. 3, implantable matrix 300 may include hydrogel matrix 302 populated with cells 404 and, optionally, seeded additives 306. Hydrogels may generally comprise three-dimensional (3D) processed (e.g., crosslinked or polymerized) networks of polymers. They are useful for tissue engineering due to their tunable biochemical and biophysical properties to control cell functions (e.g., adhesion, proliferation, differentiation, etc.) and may be designed as an artificial extracellular matrix (ECM) substrate for providing spatial orientation and promoting cellular interactions with surroundings. Hydrogels may be formed by crosslinking polymeric chains through physical or chemical methods. For example, physical hydrogels are ionotropic, formed through molecular entanglements and secondary forces such as hydrogen bonds, crystallite formation, electrostatic interactions, and hydrophobic, which are often reversible. An advantage of physical hydrogels is their biocompatible nature due to the absence of chemical cross-linkers that may cause cell toxicity.

A hydrogel matrix, such as hydrogel matrix 302, may include naturally derived biomaterials, synthetic polymers, or combinations thereof. In some variations, the hydrogel may include more than one layer of hydrogel matrix. In some variations, the hydrogel matrix 302 may be a microbead hydrogel to increase efficiency and reproducibility of large-scale production of implantable matrices described herein. In some variations, hydrogel matrix 302 may be a cell-populated hydrogel glue or adhesive that naturally adheres to an implantation site. Hydrogel glues may exhibit strong chemical interactions such as strong van der Waals interactions to achieve adhesion. A unique advantage of hydrogel glues is that they can accommodate tissue movements. In some variations, a hydrogel matrix may be an injectable hydrogel.

Any polymer that can form a hydrogel upon processing (e.g., crosslinking or polymerization) may be used for the hydrogel matrix 302 in accordance with the present invention. In some variations, polymers may be linear or branched polymers. In some variations, polymers may be dendrimers. In some variations, polymers may be homopolymers or copolymers comprising two or more monomers. Copolymers may be block copolymers, graft copolymers, random copolymers, blends, mixtures, and/or adducts of polymers. Typically, polymers in accordance with the present invention are organic polymers. In some variations, polymers may be modified with one or more moieties and/or functional groups. Any moiety or functional group can be used in accordance with the present invention. In some variations, the hydrogel may include a synthetic polymer. Advantages of synthetic hydrogels for tissue engineering applications include tunability of mechanical properties and high reproducibility at both small- and large-scale production. A synthetic environment may also permit viability as cells within the hydrogel remodel the surrounding microenvironment at an implantation site. Bioactive molecules such as proteins, enzymes and growth factors may be incorporated into a synthetic hydrogel matrix to mediate specific cell functions. Synthetic hydrogel matrices may also be chemically modified to add beneficial properties such as changing porosity and stiffness, improving stability, biocompatibility and degradability, and tuning mechanical strength for various cellular applications. Nonlimiting examples of synthetic polymers for use with the present invention include: polyglycolic acid (PGA), polylactic acid (PLA), poly(lactic-co-glycolic) acid (PLGA), poly(caprolactone) (PCL), polyethylene (PET), polycarbonate (PC), polyanhydride, polyhydroxyacid, polypropylfumarate, polycaprolactone, polyamide, polyacetal, polyether, polyester, poly(orthoester), polycyanoacrylate, polyvinyl alcohol, polyvinyl acetate (PVA), polyurethane, polyphosphazene, polyacrylate, polymethacrylate, polyurea, polystyrene, polyamine, poly(ethylene glycol), polyacrylamide (PAM), poly(aspartic acid), poly(2-hydroxy ethyl methacrylate), poly(1,3-dioxan-2-one), poly(sebacic anhydride), poly(β-hydroxyalkanoate), polycaprolactam, polylactide, polyglycolide, and any derivatives and/or combinations thereof. In some variations, the hydrogel includes a natural polymer such as carbohydrate, protein, nucleic acid, lipid, etc. Some advantages of natural hydrogels include low toxicity and high biocompatibility. Additionally, the natural environment may direct cell behavior through signaling cascades that are initiated by binding events with cell surface receptors. Further examples of natural polymers for use with the present invention include, but are not limited to, fibrin, collagen, alginate, agarose, gelatin, chitosan, dextran, hyaluronic acid, chondroitin sulphate, dermatan sulphate, keratan sulphate, heparan sulphate, and any derivatives and/or combinations thereof. In some variations, a hydrogel may include antifibrinolytic agents (e.g., fibrinogen) and/or procoagulant agents (e.g., thrombin). Such agents may be sourced from human plasma. In some variations, a natural polymer may be synthetically manufactured or partially-synthetically manufactured. In some variations, the hydrogel may include a combination of synthetic and natural polymers such as, for example, PEG and chitosan, PEG and gelatin, PEG and hyaluronic acid, PAM and gelatin, PVA and gelatin, or PAM and gelatin. In some variations, the hydrogel includes one or more polymers which have been approved for use in humans by the U.S. Food and Drug Administration. In some variations, the hydrogel matrix 302 may include a combination of the foregoing or other polymers, wherein at least one polymer is capable of forming a hydrogel. Those skilled in the art will recognize that the polymers listed herein represent an exemplary, not comprehensive, list of polymers which may be included in an implantable matrix.

Typically, a thickness of hydrogel matrix 302 may be between about 1 μm and about 5 cm, such as between about 10 μm and about 1 cm, between about 100 μm and about 5 mm, or between about 250 μm and about 1 mm. In some variations, a thinner matrix may be preferred to support oxygen-demanding seeded cells such as beta cells. In some variations, a thicker matrix may be preferred to support a larger number of seeded cells.

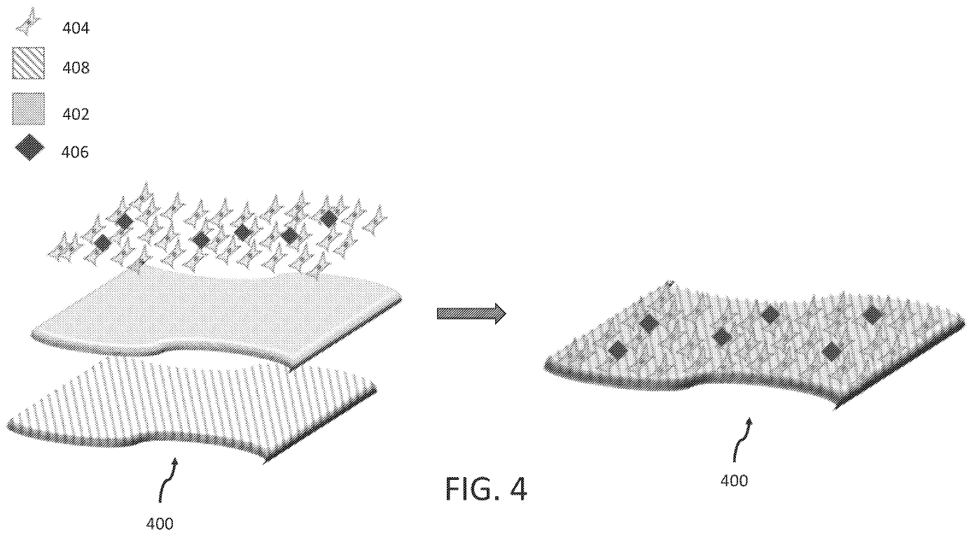

As shown in FIG. 4, in some variations, implantable matrix 400 may be a cell-populated hydrogel matrix including hydrogel 402, including cells 404 and optionally additives 406. Further, the implantable matrix 400 may be mechanically supported by mesh 408. Generally, hydrogel-mesh substrates combine the advantages of hydrogels (lubricity, biocompatibility, anti-biofouling properties, etc.) with the advantages of mesh substrates (relatively greater stiffness, toughness, strength, etc.). The mesh 408 may be fabricated from biodegradable and biocompatible polymers. Some nonlimiting examples of polymers suitable for fabricating a mesh to support a hydrogel matrix include: PLA (polylactic acid), PGA (polyglycolide), PLGA (poly(lactic-co-glycolic) copolymers, polyurethanes, polyester urethane urea (PEUU), poly(ether ester urethane) urea (PEEUU), silicones, polyaryl ether ketones, polyether ketone ketones, polyether block amides, polytetrafluoroethylene (PTFE), polyoxymethylene, polyethylene terephthalate, polypropylene polycaprolactone (PCL), poly-4-hydroxybutyrate, polycarbonate, poly(ester carbonate urethane) urea (PECUU), copolymers thereof, derivatives thereof, and combinations thereof. In some variations, a mesh may be at least partially non-polymeric and may comprise compositions including, for example and without limitation: stainless steel, gold, silver, platinum, titanium and titanium alloys, tantalum, cobalt chrome alloys, carbon fibers (graphite or diamond), hydroxyapatite and other calcium phosphate materials. In some variations, mesh 408 may be fabricated from combinations of any of the foregoing materials.

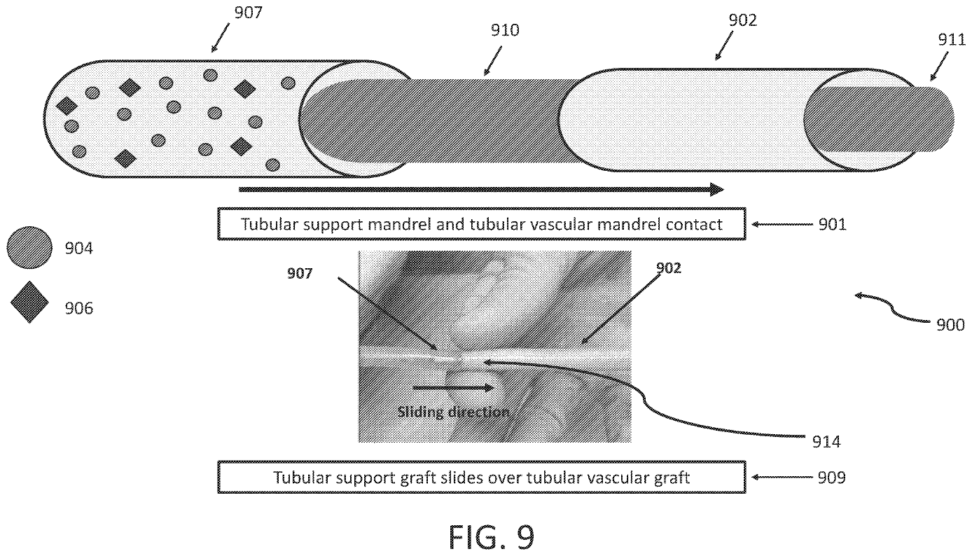

Referring again to FIG. 3, in some variations, the hydrogel matrix 302 of implantable matrix 300 may be molded into a rectangular shape. In such variations, hydrogel matrix 302 may include side lengths between about 1 mm and about 10 cm, such as between about 50 mm and about 5 cm, between about 100 mm and about 2.5 cm, between about 500 mm and about 2.25 cm, or between about 1 cm and about 2 cm. Side lengths of a rectangular hydrogel matrix may be the same or different. Alternatively, as shown in FIG. 9, which depicts a method 900 of arranging a cell-populated tubular hydrogel matrix (tubular supporting graft) concentrically around a vascular graft, the hydrogel matrix 302 may be molded into a tubular shape (e.g., a tubular support graft). In such variations, a tubular hydrogel matrix 302 may have a length between about 1 cm and 100 cm, such as between about 5 cm and about 60 cm, or between about 10 cm and about 40 cm. Tubular hydrogel substrate 308 may have an inner diameter between about 1 mm and about 50 mm, such as between about 2 mm and about 30 mm, or between about 3 mm and about 20 mm. Tubular hydrogel substrate 308 may have an outer diameter between about 1 mm and about 50 mm, such as between about 2 mm and about 30 mm, or between about 3 mm and about 20 mm. In general, an inner diameter of tubular hydrogel 308 may be greater than an outer diameter of a decellularized blood vessel such as, for example, HAV. In some embodiments, an inner and/or outer diameter of a tubular hydrogel substrate may taper (e.g., increase or decrease) from a first end to a second end. For example, tubular hydrogel matrix 302 may comprise an inner diameter of 8 mm and an outer diameter of 10 mm at a first end, and an inner diameter of 7 mm and an outer diameter of 9 mm at a second end, wherein the inner and outer diameters decrease at a constant slope from the first end to the second end.

In alternative variations, the hydrogel matrix 302 may comprise a circular shape, a triangular shape, and ovular shape, an irregular shape, or a pouch configured to contain medicament-producing cells. Those skilled in the art will recognize that the hydrogel substrate shapes listed herein represent an exemplary, not comprehensive, list of shapes suitable for a hydrogel substrate.

As shown in FIG. 1, implantable matrix 100 may be a rectangular patch including hydrogel matrix 108 attached to tissue substrate 102. Hydrogel 108 may be coated or cast onto tissue substrate 102. Typically, hydrogel 108 may be attached to an outer surface of tissue 102. In some variations, hydrogel 108 may be a hydrogel glue that may be disposed directly onto tissue substrate 102 or may be disposed onto a separate hydrogel. Methods of forming an implantable matrix including a tissue substrate, a hydrogel substrate, and a hydrogel glue are described in further detail herein. In some variations, hydrogel 108 and tissue 102 may be polymerized via methods described in further detail herein.

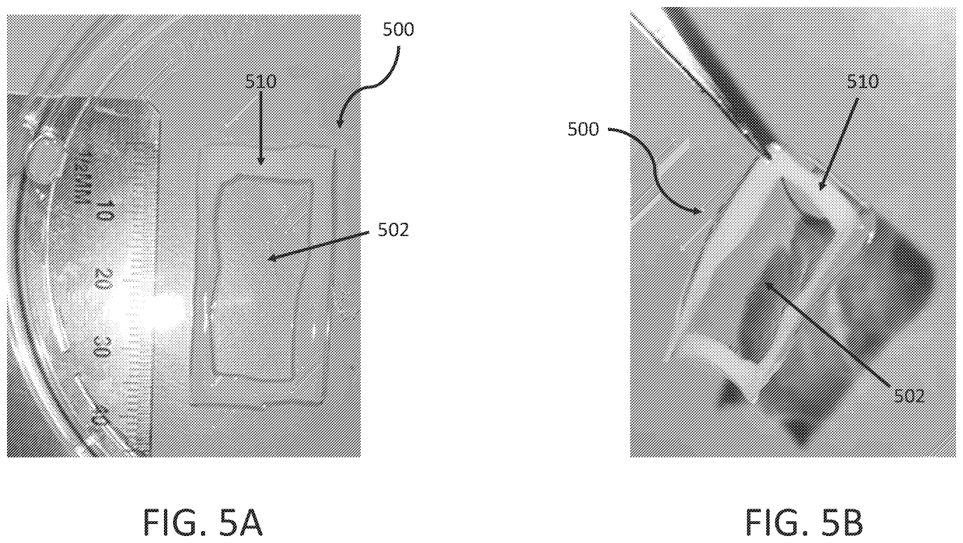

Referring to FIGS. 5A-5B, implantable matrix 500 may include a tissue substrate frame (i.e., frame 510) which surrounds hydrogel 502. Frame 510 may comprise stiff material, like collagen, to facilitate implantation (e.g., suturing, gluing, etc.) of implantable matrix 500 to an implantation site. For example, frame 510 may be a stiff, collagenous frame which may be sutured to a patient's tissue during implantation of implantable matrix 500. Frame 510 may be a rectangle, a circle, a triangle, an irregular shape, or any other suitable shape to surround hydrogel 502. A rectangular frame may include side lengths ranging between about 1 mm and 10 cm. For example, a rectangular frame may be about 1 cm×5 cm, about 2 cm×2 cm, or about 2.5 cm×2 cm.

As shown in FIG. 9, in some variations, implantable matrix 914 may be a cell-populated hydrogel sleeve 907 (e.g., tubular support graft) arranged about tubular tissue substrate 902 (e.g., tubular vascular graft). A length, a width, and/or a thickness of tubular hydrogel 907 may be greater than, less than, or equal to a length, a width, and/or a thickness of vascular graft 902. Methods of attaching vascular graft 902 and tubular support graft 907 are described in further detail herein.

For example, FIG. 9 depicts a method of arranging a cell-populated hydrogel tube concentrically around a vascular graft. The vascular graft 902 may have a diameter of between about 1 and about 20 mm, such as between about 2 mm and about 16 mm, between about 4 mm and about 12 mm, or between about 6 mm and about 8 mm. Vascular graft 902 may have a length between about 1 mm and about 10 mm, such as between about 2 mm and about 8 mm, or between about 3 mm and about 7 mm. Vascular graft 902 may have an inner diameter between about 1 mm and about 50 mm, such as between about 2 mm and about 30 mm, or between about 3 mm and about 20 mm. Vascular graft 902 may have an outer diameter between about 1 mm and about 50 mm, such as between about 2 mm and about 30 mm, or between about 3 mm and about 20 mm. In some variations, an inner and outer diameter of vascular graft 902 may be about equal to an inner and outer diameter of a decellularized blood vessel such as HAV. In some embodiments, an inner and/or outer diameter of a tubular hydrogel substrate may taper (e.g., increase or decrease) from a first end to a second end. For example, vascular graft 902 may comprise an inner diameter of 8 mm and an outer diameter of 10 mm at a first end, and an inner diameter of 7 mm and an outer diameter of 9 mm at a second end, wherein the inner and outer diameters decrease at a constant slope from the first end to the second end.

In alternate variations, a tissue substrate may comprise a circular shape, a triangular shape, and ovular shape, an irregular shape, or a pouch configured to contain medicament-producing cells. Those skilled in the art will recognize that the tissue substrate shapes listed herein represent an exemplary, not comprehensive, list of shapes suitable for a tissue substrate.

### SECTION 2: CELL SECRETION OF MEDICAMENTS AND ADDITIVES

Generally, cells for use with the present invention may secrete medicaments to treat patients with chronic diseases. Viable cells may be seeded onto and/or within any one of the matrix substrates described herein to form an implantable matrix capable of secreting medicaments. For example, as shown in FIG. 1, cells 104, and optionally additives 106, may be mixed into a hydrogel precursor solution, and hydrogel matrix 108 may be coupled to tissue substrate 102 to form implantable matrix 100. As another example, in FIG. 2, tissue substrate 202 may be surface seeded with cells 204.

Any type of cell may be used in accordance with the present invention. Nonlimiting examples of cells suitable for use with the present invention include: mammalian cells (e.g., human cells, primate cells, mammalian cells, rodent cells, etc.), avian cells, fish cells, insect cells, plant cells, fungal cells, bacterial cells, and hybrid cells. In some variations, cell types may include endothelial cells, mesenchymal stem cells, and/or hematopoietic cells such as megakaryocytes or platelets. In some variations, endothelial cells may be human liver sinusoid endothelial cells (LSEC). In some variations, cells may include monocytes. In some variations, cells may include organoids. In some variations, cells for use with the present invention may be generated from mesenchymal stem cells, endothelial progenitor cells, and/or hematopoietic stem cells. Additionally or alternatively, cells may be cultured from human endothelium including, but not limited to, tissue from artery and/or vein lining, endocardium, and/or the glomeruli of the kidneys. In some variations, exemplary cell types suitable for use with the present invention include primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g., monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc., and/or hybrids thereof, may be seeded onto and/or within a matrix in accordance with the present invention. In some variations, an implantable matrix may include cells which produce anti-inflammatory substances to alleviate the effect of infiltrating host immune cells. Anti-inflammatory substance-producing cells may include, but are not limited to, neutrophils, macrophages, monocytes, hepatocytes, lymphocytes, mast cells, and any combinations thereof. Anti-inflammatory substances may include, but are not limited to, interleukins and/or transforming growth factor-β (TGF-β). Any of the foregoing cell populations may include intracellular sources of ionized calcium. Those skilled in the art will recognize that the cells listed herein represent an exemplary, not comprehensive, list of cells that can be encapsulated within a matrix in accordance with the present invention.

In some variations, a single matrix may include a population of identical or nonidentical cell types. In some variations, a single implantable matrix may include two or more different types of cells. For example, an implantable matrix may include one or more medicament-secreting cell populations and one or more cell populations which secrete agents to suppress an immune response to the implantable matrix. Some nonlimiting examples immunosuppressant cell types include interleukin-10 expressing cells, tacrolimus-producing cells, sirolimus-producing cells, and MHC class I and II knock-out mesenchymal stem cells. Such cells may provide local protection for the medicament-producing cells in or on the implantable matrix. In some variations, a single implantable matrix may include any number of cell types. In some variations, cells may be evenly distributed throughout a matrix substrate. In some variations, cells may be distributed on the surface of a substrate. For example, cells may be distributed on an exposed surface of a tissue substrate comprising HAV. In some variations, cells may be encapsulated in the interior of a substrate, such as a hydrogel substrate. In some variations

Typically, cell populations for use with the present invention may be genetically engineered cell populations. Cells may be engineered to produce one or more medicaments such as cytokines, hormones, antibodies, proteins, fusion proteins, or any other medicament used to treat chronic diseases. For example, cells for use with the present invention may be engineered to produce one or more cytokine-, antibody-, or inhibitor-type drug molecule such as, but not limited to, Ocrelizumab, Natalizumab, Pembrolizumab, Infliximab, Vedolizumab, Dabrafenib, Lecanemab, or interferon beta-1a. Additionally or alternatively, the cells may produce or be engineered to produce produces one or more therapeutic protein, peptide, or hormone molecules such as, but not limited to, Factor VII, Factor VIII, Factor IX, Factor X, Von Willebrand factor, Protein C, human albumin, human Immune Globulin, testosterone, human Htt, or a 23aa peptide (P42). In some variations, the therapeutic proteins or peptides secreted may be anti-inflammatory. Further, in some variations, one or more proteins secreted by a cell population may be fusion proteins. Methods of cell engineering are described in further detail herein and may generally include any viral, non-viral, CRISPR-Cas9, or TALEN-transfection/gene delivery (AAV and similar)-gene delivery method. Further, in some variations, cells for use with the present invention may produce or may be engineered to produce growth factors, interferons, interleukins, chemokines, monokines, hormones, angiogenic factors, drugs, and/or antibiotics.

Any of a variety of cell culture media that can support growth of the one or more cell types or cell lines mat be used to grow and/or maintain cells in accordance with the present invention, including complex media and/or serum-free culture media. Typically, a cell culture medium contains a buffer, salts, energy source, amino acids (e.g., natural amino acids, non-natural amino acids, etc.), vitamins, and/or trace elements. Cell culture media may optionally contain a variety of other ingredients, including but not limited to, carbon sources (e.g., natural sugars, non-natural sugars, etc.), cofactors, lipids, sugars, nucleosides, animal-derived components, hydrolysates, hormones, growth factors, surfactants, indicators, minerals, activators of specific enzymes, activators inhibitors of specific enzymes, enzymes, organics, and/or small molecule metabolites.

The conditions under which cells are encapsulated within a matrix may be optimized to maximize cell viability. For example, cell viability may increase when a matrix comprises a low polymer concentration. The conditions of a cell culture medium (e.g., pH, ionic strength, nutrient availability, temperature, oxygen availability, osmolarity, etc.) may also be regulated and/or altered to maximize cell viability within a matrix. Cell viability may be measured by monitoring one or more indicators including, but are not limited to, intracellular esterase activity, plasma membrane integrity, metabolic activity, gene expression, and protein expression. For example, live cells exposed to a fluorogenic esterase substrate (e.g., calcein AM) fluoresce green as a result of intracellular esterase activity that hydrolyzes the esterase substrate to a green fluorescent product. As another example, dead cells exposed to a fluorescent nucleic acid stain (e.g., ethidium homodimer-1) fluoresce red because their plasma membranes are compromised and thus permeable to the high-affinity nucleic acid stain.

As shown in FIG. 1, implantable matrix 100 may optionally include one or more additives 106 to aid in integration of an implantable matrix into an implantation site, proliferation of cells within an implantable matrix, and/or localized therapy to treat hemophilia A or similar disorders. Additives 106 may be distributed onto or within matrix 100 during a fabrication process. Additionally, cells 104 may secrete or be engineered to secrete additives 106. Some nonlimiting examples of additives 106 include: hemostatic agents, growth factors, interferons, interleukins, chemokines, monokines, organoids, hormones, angiogenic factors, drugs, crosslinking agents, enzymes, antibiotics, antibiotics, bioelectronics, organic filler particles, and/or inorganic filler particles. For example, matrix 100 may be doped with angiogenic growth factors such as VEGF, PDGF, HGF, or FGF to facilitate vascularization in implantable matrix 100. In yet another example, matrix 100 may be loaded with inorganic and/or organic fillers to improve its mechanical properties. In some variations, matrix 100 may be doped with any suitable Fas receptor activating molecule, such as Fas ligand (e.g., CD178), to induce immune privilege for the cells in the matrix. In some variations, additives 106 may be encapsulated in biomolecules that are distributed through matrix 100 to adjust its mechanical properties and/or release of additives 106 in vivo. For example, the release rate of biomolecule-encapsulated additives from a matrix substrate may be predicted and/or controlled via enzymatic degradation of the capsules. As another example, additives 106 may include cleavable crosslinking agents or enzymes to aid in degradation of matrix 100 at the implantation site. Further, as another example, additives 106 may be bioelectronic additives that may be used to stimulate cell secretion or diffusion of other additives from matrix 100.

### SECTION 3: FABRICATION OF IMPLANTABLE MATRICES

In general, a process for making an implantable matrix may include engineering cells to produce one or more medicaments of interest and producing a 3-D matrix that is populated with engineered cells. In some variations, the one or more tissue substrates may also be seeded with cells that produce agents to protect the medicament-producing cells from an immune response. In some variations, the one or more matrix substrates may also be loaded with one or more additives described herein.

### Activating Cell Secretion

Cells may be genetically engineered produce one or more medicaments for treating chronic disease. Cell secretion of medicaments may treat chronically diseased patients systemically and/or locally at the implantation site. In some variations, cells may be engineered to additionally produce one or more of the additives described herein. In some variations, each of a plurality of cell populations within an implantable matrix may produce one or more medicament and/or one or more additive. Engineered cells may produce medicaments continuously or via initiation by a gene promotor such as demeclocycline and minocycline; cumate; tamoxifen (or other estrogen receptor drugs); tetracycline or derivatives thereof (e.g., doxycycline), or light. Any viral, nonviral, CRISPR-Cas9, or otherwise suitable gene editing technique may be used to engineer cells for medicament secretion. For example, gene targeting reagents from the CRISPR-Cas9 system may be used to target one or more genes controlling production of a medicament of interest. Gene targeting reagents from the CRISPR-Cas9 system may include Cas9 or dCas9 fused to a viral transcriptional activation domain, and the transcriptional potency of gene targeting reagents from the CRISPR-Cas9 system may be potentiated with synergistic activation modules including, for example, HSF1 and/or p65. In some variations, a gene editing technique may upregulate transcription of genes involved in medicament production and therefore an overexpression of medicaments produced by cells. Such a technique may involve, for example, a doxycycline inducible overexpression system. In some variations, upregulation of target genes may be single or multiplexed. In some variations, activation of cell populations may involve stimulation of des-amino-D-arginine vasopressin (DDAVP), resulting in cell secretion of FVIII. Further, in some variations, bioelectronics may be used to stimulate diffusion of medicaments out of an implantable matrix.

### Culturing Cells

High-quality cells in effective quantities are crucial for use in therapeutic applications like localized factor replacement therapy. Cells for use with the present invention may generally be cultured to produce a larger population of cells in a favorable artificial environment. Any of a variety of cell culture media that can support growth of the one or more cell types or cell lines may be used to grow and/or maintain cells in accordance with the present invention, including complex media and/or serum-free culture media. Cells may be cultured before reaching confluency, which may take between about 24 hours and about 1 month. For example, cells may be cultured for about 2 days, about 1 week, about 2 weeks, or about 3 weeks. In some variations, a cell culture process may be repeated more than once. A cell culture may be a 2D or 3D culture. In some variations, a cell culture may be a cell suspension. Agitation of a cell suspension may involve use of a magnetic stirrer with a culture flask or rotating spinner flasks. A cell culture medium may contain a buffer (e.g., boric acid, ammonium carbonate, calcium carbonate, sodium carbonate, citric acid, glycine, tris/glycine, ammonium phosphate, potassium phosphate, sodium phosphate, etc.), salts, energy source, amino acids (e.g., natural amino acids, non-natural amino acids, etc.), vitamins, and/or trace elements. Cell culture media may optionally contain a variety of other ingredients, including but not limited to, carbon sources (e.g., natural sugars, non-natural sugars, etc.), cofactors, lipids, sugars, nucleosides, animal-derived components, hydrolysates, hormones, growth factors, surfactants, indicators, minerals, activators of specific enzymes, activators inhibitors of specific enzymes, enzymes, organics, and/or small molecule metabolites. Typically, cell culture and environmental factors such as pH, temperature, and carbon dioxide concentrations may be optimized considering the cell line being cultured. In some variations, a cell suspension may be diluted to obtain a desired cell seeding density. In some variations, cultured cells may be immortalized to prevent senescence using techniques such as the vinyl chloride immortalization method with genes such as SV40 T antigen or human telomerase. In some variations, cells may be cryopreserved following culturing and revived (e.g., thawed) prior to mixing with a matrix. In general, a cell culture may be sampled and centrifuged to yield a cell pellet which may be mixed with a precursor solution prior to processing. Typically, a cell culture may be centrifuged for about 1 minute to about 20 minutes, such as for about 5 minutes to about 10 minutes. In some variations, a cell culture may be centrifuged and resuspended in fresh growth medium as needed throughout a cell culture process.

### Fabricating Matrices

In variations wherein an implantable matrix includes 3-dimensional tissue that is populated by cells. Methods of creating cell-populated matrices may generally involve mixing the hydrogel monomers with the cell suspension prior to polymerization of the hydrogel or pipetting cells from a culture medium to the surface of the 3-dimensional polymer network and transferring the cell-laden matrix to a growth medium.

Typically, chemical and/or mechanical tissue decellularization may be used to produce a tissue substrate. In some variations, blood vessel, skin or tendon may be decellularized to produce a tissue substrate. For example, a regenerative vascular conduit may be decellularized to produce a decellularized tissue substrate (e.g., human acellular vessel (HAV)). A decellularization process may generally include freezing and thawing of the tissue, exposing the tissue to a chemical agent, rinsing the tissue, and sterilizing the tissue. In some variations, decellularization and sterilization steps may be simultaneous. The complexity and length of the decellularization protocol may be proportional to the degree of geometric and biologic conservation desired for the post-processed tissue (e.g., macrostructure, ultrastructure, matrix and basement membrane proteins, growth factors, etc.). Typically, tissue is decellularized to an extent that avoids elicitation of a pro-inflammatory (e.g., M1 macrophage phenotype) response, which means that there is a sufficiently low concentration or amount of DNA, phospholipid, and/or mitochondrial material in the resulting tissue. In some variations, the chemical agent exposed to the tissue may include one or more of an acid, a base, a solvent (e.g., acetone, alcohols, etc.) a detergent, a hypotonic solution, or a hypertonic solution. In some variations, the tissue may further be exposed to a biological agent such as an enzyme (e.g., nucleases, trypsin, dispase, chelating agents, etc.). In some variations, a decellularization process further comprises mechanically removing undesirable tissue layers, applying of pressure to the tissue, and/or electroporating the tissue.

In some variations, shaping a decellularized tissue substrate for use with the present invention may involve cutting a tissue sheet. Particularly useful tissue substrate shapes may include rectangular patches and tubular sleeves. More nonlimiting examples of suitable shapes for a tissue substrate may include a, a circle, a triangle, an oval, or an irregular shape.

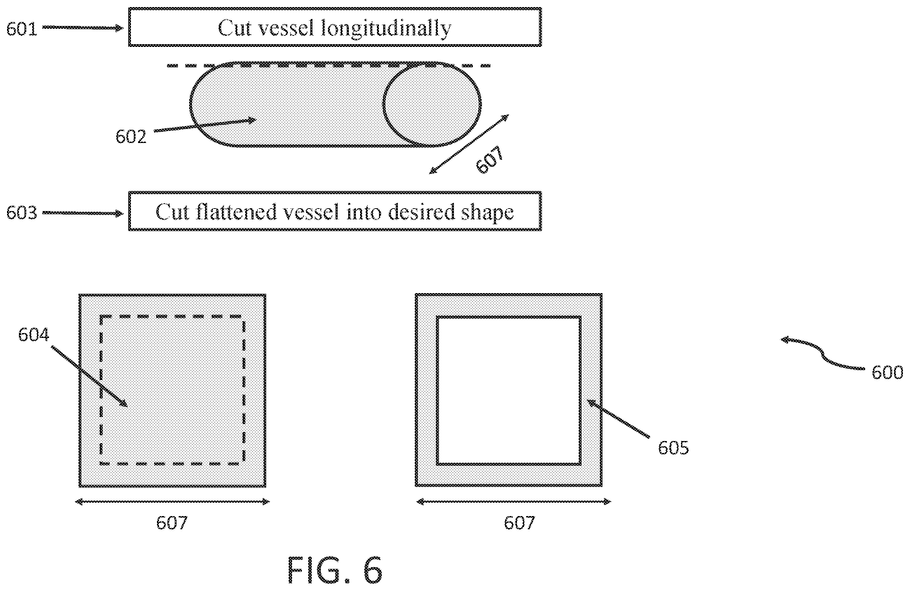

Method 600 for preparing HAV for use in an implantable matrix is shown in FIG. 6. At step 601, HAV 602 is cut along a longitudinal axis to produce flat patch 604. HAV 602 may have a length between about 1 mm and about 10 cm, such as between about 2 cm and about 8 cm, or between about 3 cm and about 7 cm. Cutting HAV 602 may involve mechanical cutting action, thermal ablation, radiofrequency ablation, cryoablation, another suitable means of cutting, or combinations thereof. Patch 604 may have a width 608 that is about equal to a circumference of HAV 602, such as between about 1 cm and about 3 cm, or between about 1.5 cm and about 2.5 cm. At step 603, a rectangular window is cut in tissue patch 604 to produce tissue frame 606 comprising an empty region in its center. In variations wherein a tissue substrate comprises an artificial acellular substrate, a precursor solution may be cast into frame-shaped mold to produce a tissue substrate frame.

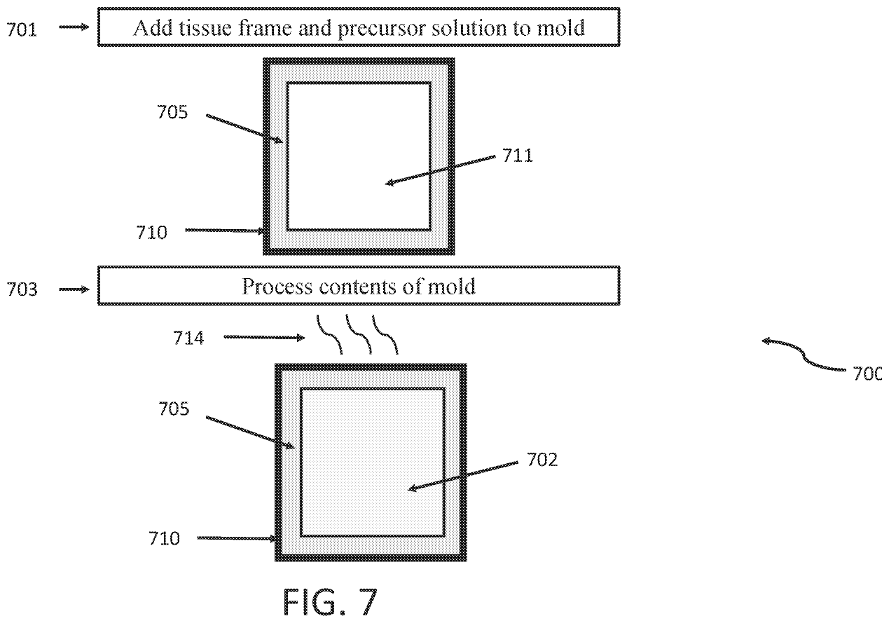

In some variations, an implantable matrix may include a matrix attached to a tissue substrate. For example, an implantable matrix may include a hydrogel substrate and a tissue substrate. FIG. 7 generally illustrates method 700 for attaching a hydrogel to a tissue frame. At step 701, frame 705 and hydrogel precursor solution 711 are cast into mold 710. At step 703, the contents of the mold undergo processing via heat source 714 such that the precursor solution forms a hydrogel matrix substrate. Any method of processing suitable to crosslink/polymerize precursor solution 711 may also cause resulting hydrogel 702 to bind to tissue frame 705. For example, frame 705 may comprise porous collagen, and the precursor solution may comprise fibrinogen and thrombin. Upon incubation, precursor solution 711 may form a fibrin hydrogel which adheres to the porous collagen tissue frame 705 via polymerization.

Generally, a hydrogel substrate may be formed via preparation of a precursor solution (e.g., solubilized matrix) and subsequent processing of the precursor solution.

A percent of prepolymer and/or polymer in the precursor solution may be one which allows for the formation of matrices via processing as described herein. In some variations, a concentration of polymer and/or prepolymer in a precursor solution may be between about 0.01 units/mL and about 1000 units/mL, such as between about 0.1 units/mL and 500 units/mL, between about 1 units/mL and about 100 units/mL, between about 10 units/mL and about 80 units/mL, or between about 20 units/mL and about 60 units/mL. For example, the concentration of polymer and/or prepolymer in a precursor solution may be between about 0.01 mg/mL and about 1000 mg/mL, such as between about 0.1 mg/mL and 500 mg/mL, between about 1 mg/mL and about 100 mg/mL, between about 10 mg/mL and about 80 mg/mL, or between about 20 mg/mL and about 60 mg/mL. As another example, a concentration of thrombin in a precursor solution may be between about 0.1 mg/mL and about 100 mg/mL and a concentration of fibrinogen in the precursor solution may be between about 3 mg/mL and about 100 mg/mL. Similarly, a percent of prepolymer and/or polymer in the precursor solution may range between about 1% w/w and about 60% w/w, between about 1% w/w and about 50% w/w, between about 1% w/w and about 40% w/w, between about 5% w/w and about 30% w/w, between about 5% w/w and about 20% w/w, or between about 5% w/w and about 10% w/w. In some variations, a percent of prepolymer and/or polymer in a precursor solution that is suitable for forming hydrogels in accordance with the present invention may be about 1% w/w, about 2% w/w, about 3% w/w, about 4% w/w, about 5% w/w, about 6% w/w, about 7% w/w, about 8% w/w, about 9% w/w, about 10% w/w, about 20% w/w, about 30% w/w, about 40% w/w, about 50% w/w, about 60% w/w, or more. In some variations, the percent of prepolymer and/or polymer in a precursor solution that is suitable for forming hydrogels in accordance with the present invention may be approximately 5% w/w. In general, the absorption capacity of a hydrogel matrix increases with higher polymer concentrations in a precursor solution.

In some variations, a precursor solution may include a buffer such as boric acid, ammonium carbonate, calcium carbonate, sodium carbonate, citric acid, glycine, tris/glycine, ammonium phosphate, potassium phosphate, sodium phosphate, or any other suitable buffer. In some variations, a precursor solution may include a dispersing agent. In some variations, a precursor solution may include one or more firming agent such as calcium carbonate, calcium hydrogen sulfite, calcium citrates, calcium phosphates, calcium chloride, magnesium chloride, magnesium sulfate, calcium gluconate, or magnesium gluconate. In some variations, a precursor solution prepared may include a first polymer and/or prepolymer and a second polymer and/or prepolymer, wherein the concentrations of the first and second polymers and/or prepolymers may be different. In some variations, a precursor solution may comprise a plurality of polymers and/or prepolymers, each having a different concentration. In some variations, a precursor solution may include more than one precursor solutions. In some variations, each of a plurality of precursor solutions prepared may include different prepolymers and/or polymers, or different concentrations of prepolymers and/or polymers.

A precursor solution may be mixed with cells prior to processing. Generally, a prepared precursor solution may be kept on ice or in a temperature-controlled environment prior to and/or during mixing with cells. Cells may be seeded into a precursor solution using any suitable method. For example, a pipette may be used to transfer cells to a flask containing a precursor solution. Then, the flask contents may be pipetted up and down to mix the solution for easy seeding. In some variations, a precursor solution may be sterilized prior to mixing with cells. A sterilization process may include filtration techniques such as membrane filtration, Seitz filtration, sintered glass filtration, or candle filtration. In some variations, two or more discrete precursor solutions may be filtered independently and subsequently mixed. In alternative variations, a hydrogel substrate may be surface seeded with cells after processing, or before processing and after processing.

In general, the percent of cells in a precursor solution may be one that allows for the formation of matrices in accordance with the present invention. In some variations, the percent of cells in a precursor solution may be between about 0.1% w/w and about 80% w/w, between about 1.0% w/w and about 50% w/w, between about 1.0% w/w and about 40% w/w, between about 1.0% w/w and about 30% w/w, between about 1.0% w/w and about 20% w/w, between about 1.0% w/w and about 10% w/w, between about 5.0% w/w and about 20% w/w, or between about 5.0% w/w and about 10% w/w. In some variations, the percent of cells in a precursor solution that is suitable for forming matrices in accordance with the present invention may be approximately 5% w/w. In some variations, the concentration of cells in a precursor solution that is suitable for forming matrices in accordance with the present invention may range between about 1×10⁵ cells/mL and 1×10⁸ cells/mL or between about 1×10⁶ cells/mL and 1×10⁷ cells/mL.

The conditions under which cells are encapsulated within a hydrogel may be optimized to maximize cell viability. For example, cell viability may increase when a matrix comprises a low polymer concentration. The conditions of a cell culture medium (e.g., pH, ionic strength, nutrient availability, temperature, oxygen availability, osmolarity, etc.) may also be regulated and/or altered to maximize cell viability within a matrix. Cell viability may be measured by monitoring one or more indicators including, but are not limited to, intracellular esterase activity, plasma membrane integrity, metabolic activity, gene expression, and protein expression. For example, live cells exposed to a fluorogenic esterase substrate (e.g., calcein AM) fluoresce green as a result of intracellular esterase activity that hydrolyzes the esterase substrate to a green fluorescent product. As another example, dead cells exposed to a fluorescent nucleic acid stain (e.g., ethidium homodimer-1) fluoresce red because their plasma membranes are compromised and thus permeable to the high-affinity nucleic acid stain.

A precursor solution or cell-laden precursor solution may be cast into a mold such that an implantable matrix forms to the shape of the mold upon processing. A mold may be any suitable 3D shape such as a rectangular prism, a hexagonal prism, a pentagonal prism, a cylinder, etc. A mold may comprise any material. In some variations, a greatest dimension of a mold may be between about 1 μm and about 10 m. For example, a greatest dimension of a mold may be between about 100 μm and about 1 m, between about 1 mm and about 10 cm, or between about 100 mm and about 1 cm. In some variations, a mold may be treated prior to use to alter its surface properties. For example, a mold may be made hydrophilic via plasma cleaning, chemical derivatization of surface, or any other suitable method. In some variations, a mold may be treated with a bioreagent such as a surfactant.

In some variations, an implantable matrix may be formed via 3D printing or electrospinning. For example, a cell-laden hydrogel may be used as ink by a 3D printer to print an implantable matrix. As another example, a 3D printer may be used to deposit cell seeded hydrogel glue onto a substrate.

A precursor solution or cell laden precursor solution may be processed to form an implantable matrix. Processing a precursor solution may be achieved by physical or chemical methods and may be controlled by a variety of environmental factors such as temperature, pH, and/or the addition of chelating ions. In some variations, a precursor solution may include one or more enzymes to aid in processing. For example, a precursor solution may include fibrinogen and thrombin, which is crucial to enzymatic polymerization of fibrinogen, to form a fibrin hydrogel. In some variations, processing of a precursor solution may include thermal curing. For example, a precursor solution may process via incubation in a temperature-controlled environment for an appropriate amount of time. A temperature-controlled environment may have a temperature between about −15° C. and about 100° C., such as between about 0° C. and about 60° C., between about 15° C. and about 50° C., or between about 30° C. and about 40° C. For example, a precursor solution containing thrombin and fibrinogen may be incubated at a temperature of about 37° C. A predetermined amount of time may be between about 1 second and 10 days, such as between about 30 seconds and about 10 hours, between about 10 minutes and about 12 hours, or between about 15 minutes and about 1 hour. For example, a precursor solution containing thrombin and fibrinogen may be incubated for about 20 minutes. As another example, a hydrogel glue precursor solution may undergo curing for hours to days to establish strong cohesion and interfacial adhesion. In some variations, processing of a precursor solution may include photopolymerization in the presence of photoinitiators via exposure to ultraviolet (UV) light. In some variations, a processing process may include irradiation with B, y or X radiation. In some variations, processing may include applying repeated freezing-thawing cycles to the precursor solution. In some variations, processing may include using chemical agents such as di-aldehydes or di-halide derivatives of hydrocarbons.

In some variations, fabricating a hydrogel matrix may include stacking layers of hydrogel throughout a processing process. Stacking may be achieved by any suitable method and may include any number of hydrogel layers. For example, stacking hydrogel layers may involve adding a first precursor solution to a mold, at least partially crosslinking the first precursor solution, adding a second precursor solution to the mold, and at least partially crosslinking the stacked solutions within the mold.

In some variations, fabricating an implantable matrix may include depositing a hydrogel matrix onto a mesh matrix. A general method of depositing a hydrogel onto a mesh substrate may include casting a hydrogel precursor solution onto a mesh substrate within a mold and subsequently processing the contents of the mold to attach the two substrates. Typically, the hydrogel may gel around the mesh. In some variations, prior to step 1, the mesh may be treated with molecules which covalently bond to both the mesh substrate and the hydrogel, resulting in a strong bond between the two. Molecules used to treat the mesh may include, for example, bridge molecules (e.g., silanes or silane-coupling agents such as (3-aminopropyl)triethoxysilane (APTES) and 3-(trimethoxysilyl) propyl methacrylate (TMSPMA)). Alternatively, the mesh substrate may be primed with a mixture including a photo-initiator (e.g., benzophenone) and processing may involve curing the precursor solution with UV light, resulting in the grafting of hydrogel polymer chains to the mesh network. In some variations, the precursor solution may be painted onto the mesh. Painting may be achieved using any suitable painting technology. The hydrogel solution may include copolymers of hydrogel monomers and coupling agents and may behave like a viscous liquid or a common paint. The functional groups on the coupling agents may have tunable kinetics to interact with each other and with the complementary functional groups on the mesh substrate for hydrogel paint curing and strong bond formation after applying the hydrogel paint to the mesh

In some variations, an injectable hydrogel substrate may be stabilized by physical cross-linking including hydrogen bonds, hydrophobic interactions, ionic interactions, ligand-ion coordination, dipole-dipole interactions, and host-guest complexations. Physically crosslinked hydrogels usually provide a friendly environment to cells and bioactive molecules. They generally show a relatively low mechanical strength, and their morphology and properties are easily altered in external stimuli. On the other hand, the chemically crosslinked hydrogels formed by irreversible covalent bonds have shown the relatively high mechanical strength and stability. In some variations, an injectable hydrogel substrate may be stabilized by physical crosslinking and further reinforced by limited chemical crosslinking (e.g., Diels-Alder reactions, Michael additions, Schiff base reactions, enzyme-mediations, photopolymerizations, etc.). For example, a complexing agent such as cyclodextrin may be added to a hydrogel precursor solution to couple to the prepolymer/polymer via host-guest interactions, and UV-initiated polymerization of the complexing agent may subsequently be used to reinforce the hydrogel network. In some variations, an injectable hydrogel substrate may be further processed by external stimuli after the implantable matrix is injected into the user. External stimuli may include temperatures, pHs, lights, electric/magnetic fields, ultrasounds, biomolecular species such as enzymes, etc. For example, a user's body temperature may induce further crosslinking in an implantable matrix within the user.

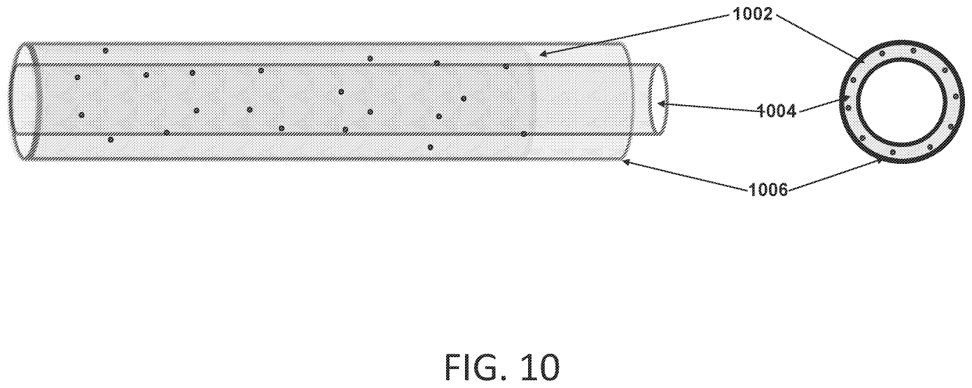

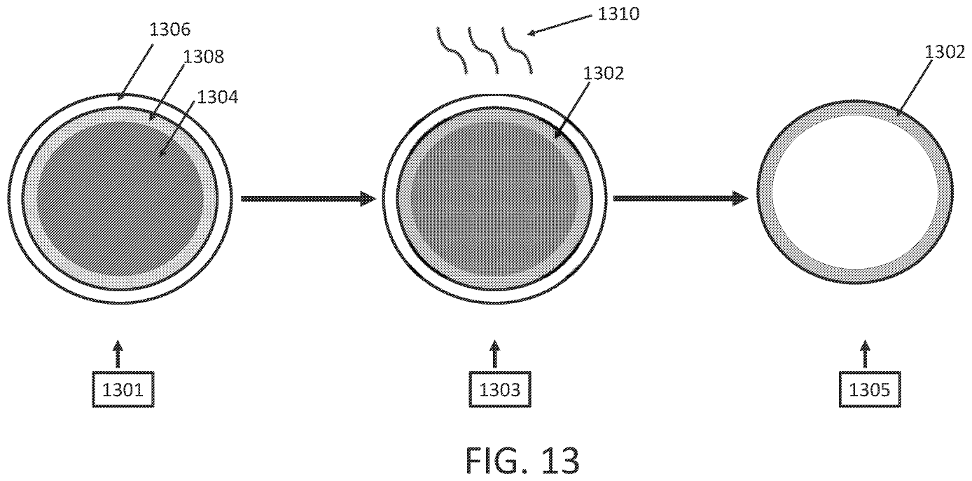

In some variations, a cell-populated hydrogel matrix may be a tubular support graft (i.e., tubular hydrogel matrix), which may be arranged concentrically over a tubular vascular graft (i.e., tubular tissue substrate). FIG. 10 shows tubular support graft 1000, which includes cell-seeded hydrogel matrix 1002. The hydrogel matrix 1002 is tubular, and was formed between mold mandrel rod 1004 and mold 1006. FIG. 13 shows consecutive cross-sectional views of a tubular support graft during a fabrication process. At step 1301, mandrel rod 1304 is inserted into a mold 1306 and hydrogel precursor solution and cell mixture 1302 is subsequently cast into mold 1306. Nonlimiting examples of materials for the mold may include glass, silicone, plastic, and metal. Typically, mold 1306 may be treated before use with, for example, Pluronic treatment. Mandrel rod 1304 may have an outer diameter of between about 1 mm and about 10 cm, such as between about 2 mm and about 1 cm, between about 3 mm and about 50 mm, between about 4 mm and about 10 mm, or between about 5 mm and about 8 mm. In some variations, a plug may be used to seal a first end of mold 1306 and center mandrel rod 1304 inside of mold 1306 prior to casting precursor solution 908 into mold 1306. Precursor solution and cell mixture 1302 may include, for example, a fibrinogen, thrombin, Calcium, and cells. In some variations, a plug may be used to seal a second end of mold 1306 and center mandrel 1304 completely inside of mold 1306. At step 1303, mold 1306 and its contents receive heat from heat source 1310 (e.g., are incubated) to polymerize precursor solution 1302 and produce cell-populated tubular hydrogel (tubular support graft) 1302. Processing via incubation may take between about 1 minute and about 1 hours, such as between about 5 minutes and about 1 hour, or between about 15 minutes and about 20 minutes. Incubation may take place in a temperature-controlled environment having a temperature of between 20° C. and about 40° C., such as between about 25° C. and about 38° C., between about 22° C. and about 40° C., or between about 30° C. and about 37° C. At step 1305, mandrel rod 1304 is removed from mold 1306 to release polymerized tubular support graft 1302 from the mold. In some variations, tubular support graft 1302 may be transferred to a final mandrel that may be larger than mandrel rod 1304 used during initial fabrication of tubular support graft 902. For example, initial support mandrel 1304 may have an outer diameter of about 7 mm, and the final support mandrel may have an outer diameter of about 8 mm.

In alternative embodiments, tubular support graft and/or the tubular vascular graft may be fabricated via electrospinning a tubular matrix and subsequently seeding with cells, and/or 3-dimension printing or bioprinting, casting, molding, planar cell culture, folding/suturing, and the like.

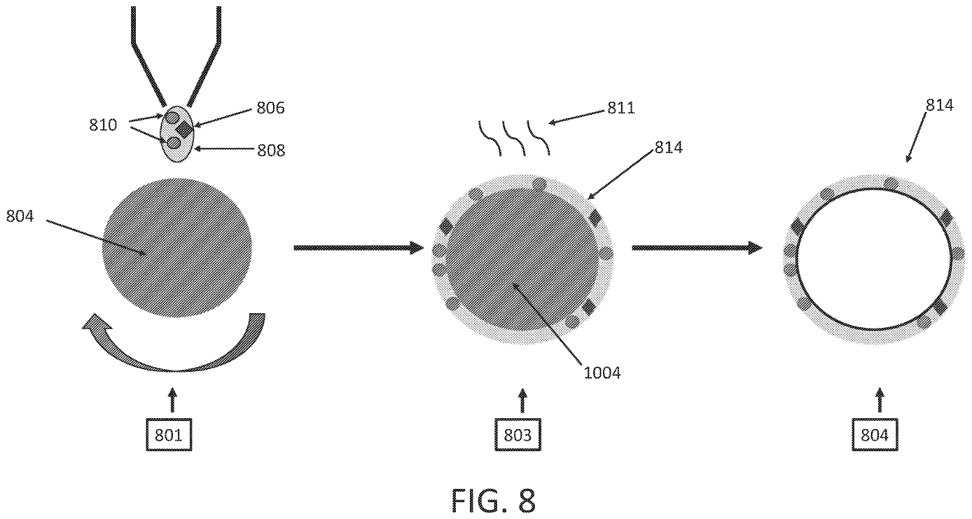

For example, tubular support graft can be created by 3D printing a bioink 808 comprising cells 810 (and optionally additives 806) onto a support mandrel 804. FIG. 8 shows consecutive cross-sectional views of method 800 for fabricating a tubular support graft. At step 801, tubular support graft 802 (i.e., cell-populated tubular hydrogel) is deposited onto the support mandrel rod 804, mandrel 804 is rotated at a predetermined speed, and hydrogel glue (bioink) 808, comprising cells 810, and optionally additives 806), is deposited onto the rotating mandrel 804. Support mandrel 804 may have an outer diameter of between about 1 mm and about 10 cm, such as between about 2 mm and about 1 cm, between about 3 mm and about 50 mm, between about 4 mm and about 10 mm, or between about 5 mm and about 8 mm. The predetermined speed may be between about 1 rpm and about 1,000 rpm, such as between about 10 rpm and about 200 rpm, between about 30 rpm and about 150 rpm, or between about 50 rpm and about 100 rpm. Nonlimiting examples for depositing hydrogel glue 808 may include pipetting, pouring, or 3D printing the cell laden hydrogel glue onto the surface of the mandrel. In some variations, 3D printing a cell laden hydrogel glue may include using s 3D bioprinter loaded with the cell laden hydrogel glue. At step 803, tubular support graft 814 is cured via heat source 812. At step 805, tubular support graft 814 is removed from support mandrel 804 and stored until use. In some variations, tubular support graft 814 may be stored on a final support mandrel that may be larger than support mandrel 804 used during initial fabrication of the tubular support graft, which may facilitate arranging the tubular support graft concentrically around a tubular vascular graft (i.e., tubular tissue substrate). In general, prior to implantation of an implantable matrix including a tubular support graft and tubular vascular graft, the tubular support graft may be concentrically positioned around a tubular vascular graft prior to implantation of the construct.

FIG. 9 illustrates a method of arranging the tubular support graft around the tubular vascular graft. At step 901, final tubular support mandrel 910, over which tubular support graft 907 is arranged, contacts vascular mandrel 911, over which tubular vascular graft 902 is arranged. Tubular support graft 907 may include medicament-secreting cells 904, and optionally additives 906. In some variations, vascular mandrel 911 may be at least partially inserted into a lumen of final tubular support mandrel 910 to arrange tubular support graft 907 over tubular vascular graft 902. At step 903, tubular support graft 907 is slid off the final support mandrel and onto and across an outer surface of the tubular vascular graft 902, resulting in biovascular medicament-producing implantable matrix.

Any one of the preceding matrix substrates may be doped with additives described herein. Generally, doping or loading a matrix may involve submersing a matrix in an aqueous solution comprising an additive, or dispersing an additive into a precursor solution. For example, an implantable matrix may be doped with tissue thromboplastin to promote coagulation at the implantation site. As another example, an implantable matrix may be doped with angiogenic growth factors such as VEGF, PDGF, HGF, or FGF to facilitate vascularization in the matrix. In some variations, the release rate of loaded additives may be determined by the diffusion of the additives into tissue surrounding the implantation site and may be accelerated using a biodegradable hydrogel matrix. For example, the drug release rate may be tuned by tailoring the molecular interactions between a hydrogel and one or more loaded additives (e.g., covalent linkage, electrostatic interaction, hydrophobic interaction). In some variations, loading a matrix with inorganic and/or organic fillers may adjust the mechanical properties of the matrix. Filler particles may be retained in an implantable matrix via physical and/or chemical bonds or by mechanical immobilization. A physical or chemical interaction between a polymer matrix and a filler particle may lead to more stable composite systems.

### Culturing Implantable Matrices

An implantable matrix may be stored in a growth medium to sustain and allow proliferation of cells until implantation. In some variations, a cell culture medium may be disposed onto an implantable matrix within a mold. In some variations, an implantable matrix that was fabricated using a mold may be removed from the mold and placed into a separate container containing a cell culture medium. In some variations, only part of an implantable matrix may be cultured after fabrication. For example, an implantable matrix may include a cell-populated tubular support graft and a tubular vascular graft, but only the tubular support graft may require culturing until implantation. In this example, the cultured, tubular support graft may be attached to or slid onto the tubular vascular graft just prior to implantation.

### Section 4: Implantation of Implantable Matrices

Described below is an overview of various aspects of a method of use of an implantable matrix.

As described above, an implantable matrix may be attached to an implantation site of interest of a user such that the seeded cells may secrete medicaments to treat chronic disease systemically within a patient and/or locally at the implantation site. A general method of implantation of a matrix may include surgically accessing an implantation site and attaching a matrix to the implantation site. Any suitable surgical means, including invasive and minimally invasive surgical techniques, may be used to access the implantation site. In some variations, more than one implantation site may be accessed within a patient. An implantation site may include a at least a portion of a tendon, an organ (e.g., stomach, intestines), a muscle, a joint (e.g., knee, hip, elbow, wrist), blood vessel, extremity (e.g., arm, leg) or any internal site where treatment is needed in a patient. Generally, implantable matrices described herein may be sutured, glued, injected into, or attached by any other suitable means to an implantation site.

In some variations, glue used to attach an implantable matrix to an implantation site may be a biodegradable and absorbable agent such as fibrin glue or a cyanoacrylate (e.g., cyanoacrylic acid).

In variations wherein an implantable matrix includes a tissue substrate, the matrix may sutured, glued, or otherwise attached to the implantation site via the tissue substrate. For example, an HAV frame surrounding a cell-laden hydrogel matrix may be used to suture the implantable matrix to the implantation site.

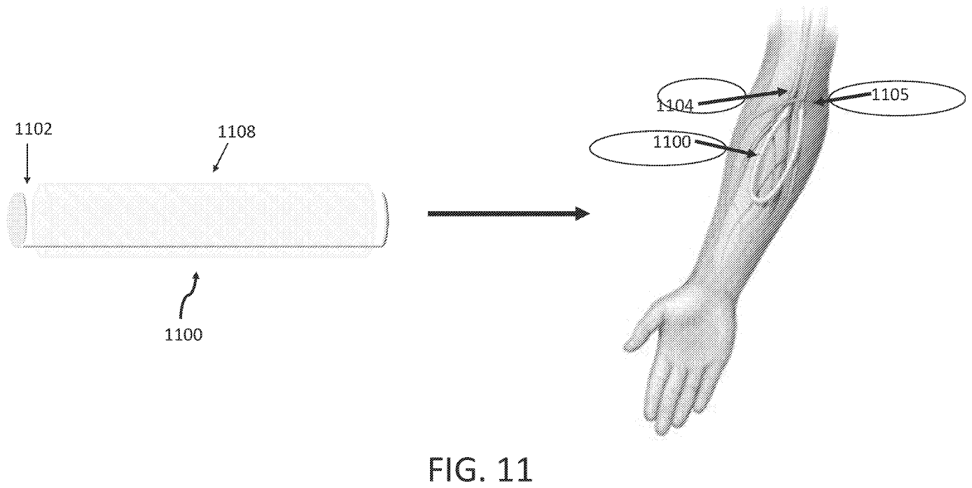

As shown in FIG. 11, implantable matrix 1100 including vascular support graft 1102 may be anastomosed to one or more blood vessels (e.g., artery 1104, vein 1106) of a patient such that the medicaments secreted by the cells are proximal to the patient's bloodstream and may be easily circulated throughout the patient's body for systemic therapy. For example, cells may be seeded in or on coating 1108 and may be engineered to secrete IFN-Beta-1a and/or Ocrelizumab to treat multiple sclerosis systemically within the patient.

In variations wherein an implantable matrix includes an injectable hydrogel substrate, the implantable matrix may be injected into an implantation site. In some variations, an injectable implantable matrix may be delivered to an irregularly shaped site. In some variations, an injectable implantable matrix may conform to the shape of a user's implantation site upon and/or after implantation. In some variations, an implantation site may include a cartilaginous joint or synovial joint. In some variations, injecting an implantable matrix with an injectable hydrogel substrate may be a minimally invasive procedure. In some variations, injecting an implantable matrix with an injectable hydrogel substrate may involve using a syringe.

In variations wherein an implantable matrix includes a functionalized tubular support graft and a tubular vascular graft, the functionalized tubular support graft may be attached to or slid onto the tubular vascular graft just prior to implantation. In some variations, a tubular support graft may surround a tubular vascular graft. In some variations, a combined tubular support graft and tubular vascular graft may be polymerized via any suitable polymerization mechanism prior to implantation.

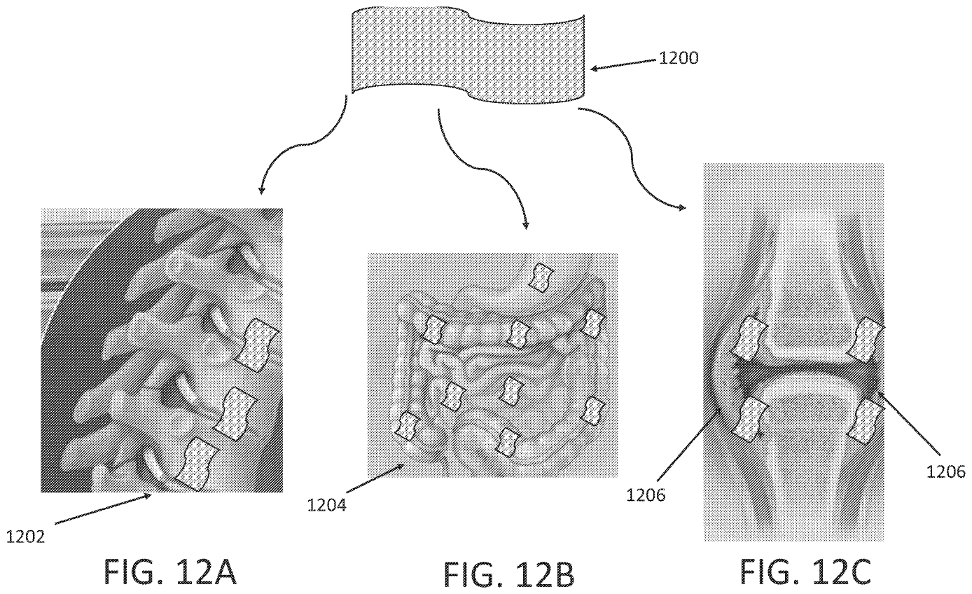

Any number of implantable matrices comprising medicament-secreting cells to treat one or more chronic diseases may be implanted at any number of implantation sites within a user. More than one type of attachment mechanism may be used to attach an implantable matrix to an implantation site. In some variations, more than one implantable matrix may be implanted per site. For example, as show in FIGS. 12A-12C, several implantable matrices 1200 may be attached to implantation sites of interest, including vertebrae 1202, intestines 1204, and joint linings 1206. Therapeutic implantable matrices for implantation on vertebrae 1202 and/or joint linings 1206 may treat chronic inflammation with seeded anti-inflammatory drug-producing cells. Therapeutic implantable matrices for implantation in and/or on the intestines may treat Crohn's disease with seeded Infliximab- and/or Vedolizumab-producing cells.

In variations wherein an implantable matrix includes a hydrogel glue, the implantable matrix may adhere to an implantation site without the use of an additional attachment mechanism.

One or more surgical instruments may be used during implantation. Exemplary surgical instruments include, but are not limited to, scissors, blades, forceps, clamps, needles, suction tubes, clips, cameras, cauterizers, wrenches, depth probes, retractors, gauge indicators, and chisels. Additionally, one or more delivery devices may be used to implant an implantable matrix. A delivery device may generally contain or grip an implantable matrix during implantation. Some nonlimiting examples of delivery devices include syringes, tubes, and clips. In some variations, delivery devices may be externally operable. For example, a clip may grip an implantable matrix as it is delivered to an implantation site and may be actuated by a handle that remains outside of a patient's body during implantation.

### SECTION 5: EXAMPLES

### Example 1: Direct Coating of Hydrogel Seeded with Medicament-Producing Cells onto a Vascular Graft

Fibrinogen powder from human was dissolved in PBS or culture medium at 37° C. for 1-2 hours until the solution was transparent with desired fibrinogen concentration of about 40.9 mg/mL. Next, the fibrinogen solution was filter sterilized in a biosafety cabinet. Sterile Calcium Chloride solution was added to the fibrinogen solution and the mixture was placed on ice. Also, thrombin solution from human plasma was placed on ice in a separate conical tube. Drug-producing cells were collected from a culture flask and centrifuged at 200 rcf for 3 minutes. The supernatant was discarded, and the islet pellet was gently resuspended in fibrinogen solution on ice. Thrombin solution was added to the fibrinogen solution and mixed well by pipetting the fibrinogen solution up and down, on ice. Next, a glass tubular mold with an inner diameter of 8 mm was capped from one end with a silicone plug and filled with sterile distilled water with Pluronic F127. Pluronic treatment was applied for 30 minutes and the mold was briefly rinsed with distilled water. A cylindrical mandrel with 5 mm outer diameter was inserted into the lumen an HAV vascular graft. The vascular graft with the mandrel in its lumen was inserted into the glass tubular mold. The cell/fibrinogen/CaCl/thrombin mixture was be cast into the space between the outer surface of the vascular graft and the glass mold. The entire system was placed into a sterile closed container and incubated at 37° C. for 15-20 minutes to polymerize the mixture. After the polymerization, the vascular graft with drug-producing cell carrying hydrogel coating was removed from the mold and transferred into a bottle containing a preferred culture medium.

### Example 2: Vascular Support Draft Directly Seeded with Medicament-Producing Cells

Fibrinogen powder from human was dissolved in PBS or culture medium at 37° C. for 1-2 hours until the solution was transparent with desired fibrinogen concentration of about 40.9 mg/mL. Next, the fibrinogen solution was filter sterilized in a biosafety cabinet. Sterile Calcium Chloride solution was added to the fibrinogen solution and the mixture was placed on ice. Also, thrombin solution from human plasma was placed on ice in a separate conical tube. Drug-producing cells were collected from a culture flask and centrifuged at 200 rcf for 3 minutes. The supernatant was discarded, and the islet pellet was gently resuspended in fibrinogen solution on ice. Thrombin solution was added to the fibrinogen solution and mixed well by pipetting the fibrinogen solution up and down, on ice. Next, a glass tubular mold with an inner diameter of 8 mm was capped from one end with a silicone plug and filled with sterile distilled water with Pluronic F127. Pluronic treatment was applied for 30 minutes and the mold was briefly rinsed with distilled water. A mandrel rod with 7 mm outer diameter was inserted into the glass mold. A piece of silicone tubing was used between the glass mold and the mandrel rod to seal one end of the mold and to center the mandrel mold inside the mold. The drug-producing cell/fibrinogen/CaCl/thrombin mixture was cast into the space between the glass mold and the mandrel rod quickly. The second end of the glass mold was sealed, and the mandrel rod was centered completely with a second segment of silicone tubing. The mold and its contents were placed into a sterile closed tray and incubated at 37° C. for 15-20 minutes to polymerize the fibrin gel. After polymerization, the drug-producing-cell-populated fibrin sleeve was removed from mold by removing the mandrel rod. The drug-producing-cell-populated fibrin sleeve was first transferred to a new mandrel rod with an outer diameter of 8 mm by gently sliding it from original 7 mm mandrel to the 8 mm mandrel. Transferring the sleeve to a larger mandrel facilitates sliding the sleeve onto an HAV with an approximate outer diameter of 7 mm. Then, the 8 mm mandrel carrying the sleeve was placed in a flask of culture medium and stored at 4° C. until the implantation of the sleeve.

### Example 3: Attachment of Hydrogel Seeded with Medicament-Producing Cells to a Vascular Support Graft Frame

Medicament-producing patches can be created with hydrogels that are populated with VWF secreting cells. A rectangular prism-shaped mold was treated with Pluronic F-127 for 30 minutes and then briefly rinsed with distilled water.

Fibrinogen powder from human plasma was dissolved in PBS or culture medium at 37° C. for 1-2 hours until the solution was transparent with a fibrinogen concentration of about 40.9 mg/mL. Then the fibrinogen solution is filter sterilized in a biosafety cabinet. Sterile Calcium Chloride solution was added to the fibrinogen solution and the mixture was placed on ice. Also, thrombin solution from human plasma was placed on ice in a separate conical tube.

Medicament-secreting cells were collected from the culture flask and centrifuged at 500 rcf for 5 minutes. The supernatant was discarded, and the cell pellet was gently resuspended in fibrinogen solution on ice. Thrombin solution was added to the fibrinogen solution and mixed well by pipetting the fibrinogen solution up and down, on ice. The cell/fibrinogen/CaCl/thrombin mixture was cast into the mold quickly. The mold and its content were placed into a sterile closed tray and incubated at 37 C for 15-20 minutes to allow the polymerization of fibrin gel. After the processing period, the medicament-secreting cell-populated fibrin gel sheet was removed from mold and transferred into a cell culture flask with culture medium.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that any and all parameters, dimensions, materials, and configurations described herein are meant to be an example and that the actual parameters, dimensions, materials, and/or configurations will depend upon the specific application or applications for which the inventive teachings disclosed herein is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the claims supported by the disclosure, and equivalents thereto, inventive embodiments may be practiced otherwise than as specifically described and claimed. Inventive embodiments of the present disclosure are also directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Embodiments disclosed herein may also be combined with one or more features, as well as complete systems, devices and/or methods, to yield yet other embodiments and inventions. Moreover, some embodiments, may be distinguishable from the prior art by specifically lacking one and/or another feature disclosed in the particular prior art reference(s); i.e., claims to such embodiments are distinguishable from the prior art by including one or more negative limitations.

Also, various inventive concepts may be embodied as one or more methods. The acts performed as part of the method may be ordered in any suitable way. Accordingly, embodiments may be constructed in which acts are performed in an order different than illustrated, which may include performing some acts simultaneously.

The indefinite articles “a” and “an,” as used herein in the specification and in the claims, unless clearly indicated to the contrary, should be understood to mean “at least one.” The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A medicament-producing implantable matrix comprising a matrix comprising at least one population of cells.

2. The implantable matrix of claim 1, wherein the at least one population of cells produces one or more drug molecules selected from the group consisting of: Ocrelizumab, Natalizumab, Pembrolizumab, Infliximab, Vedolizumab, Dabrafenib, Lecanemab, or interferon beta-1a.

3. The implantable matrix of claim 1, wherein the at least one population of cells produces one or more molecules selected from the group consisting of: Factor VII, Factor VIII, Factor IX, Factor X, Von Willebrand factor, Protein C, human albumin, human Immune Globulin, testosterone, human Htt, or P42.

4. The implantable matrix of claim 1, wherein the at least one population of cells produces interleukin-10.

5. The implantable matrix of claim 1, wherein the at least one population of cells produces one or more of a sirolimus or a tacrolimus.

6. The implantable matrix of claim 1, wherein the at least one population of cells comprises mesenchymal stem cells.

7. The implantable matrix of claim 1, wherein the at least one population of cells comprises major histocompatibility complex class I and II knock-out mesenchymal stem cells.

8. The implantable matrix of claim 1, wherein the matrix comprises at least one selected from the group consisting of: a hydrogel and a biodegradable polymer.

9. The implantable matrix of claim 1, wherein the matrix comprises a Fas receptor activating molecule.

10. The implantable matrix of claim 9, wherein the Fas receptor activating molecule comprises a Fas ligand.

11. The implantable matrix of claim 1, wherein the matrix comprises a hydrogel comprising thrombin and fibrinogen.

12. The implantable matrix of claim 11, wherein the thrombin is at a concentration of between about 0.1 units/mL and about 100 units/mL.

13. The implantable matrix of claim 11, wherein the fibrinogen is at a concentration of between about 3 units/mL and about 100 units/mL.

14. The implantable matrix of claim 1, wherein the matrix is implanted into at least one body region of a patient selected from the group consisting of: arms, legs, knees, hips, elbows, wrists, spine, stomach, blood vessels, and intestines.

15. The matrix of claim 1, wherein the matrix can be formed in the desired implantation site by injecting the cells in a hydrogel glue solution.

16. The implantable matrix of claim 8, wherein the biodegradable polymer is one selected from the group consisting of: polyglycolic acid, polylactic acid, poly(lactic-co-glycolic) acid, poly(caprolactone).

17. The implantable matrix of claim 8, wherein the hydrogel is at least one of a naturally-derived hydrogel and a synthetic hydrogel.

18. A medicament-producing implantable matrix, comprising:
a frame comprising collagen; and
a matrix comprising at least one population of cells, wherein the matrix is at least one of attached to and within the frame.

19. The implantable matrix of claim 18, wherein the at least one population of cells produces one or more molecules selected from the group consisting of: Ocrelizumab, Natalizumab, Pembrolizumab, Infliximab, Vedolizumab, Dabrafenib, Lecanemab, or interferon beta-1a

20. The implantable matrix of claim 18, wherein the at least one population of cells produces one or more molecules selected from the group consisting of: Factor VII, Factor VIII, Factor IX, Factor X, Von Willebrand factor, Protein C, human albumin, human Immune Globulin, testosterone, human Htt, or P42.

21. The implantable matrix of claim 18, wherein the at least one population of cells produces interleukin-10.

22. The implantable matrix of claim 18, wherein the at least one population of cells produces one or more of a sirolimus or a tacrolimus.

23. The implantable matrix of claim 18, wherein the at least one population of cells comprises mesenchymal stem cells.

24. The implantable matrix of claim 18, wherein the at least one population of cells comprises major histocompatibility complex class I and II knock-out mesenchymal stem cells.

25. The implantable matrix of claim 18, wherein the matrix comprises at least one selected from the group consisting of: a hydrogel and a biodegradable polymer.

26. The implantable matrix of claim 25, wherein the biodegradable polymer is one selected from the group consisting of: polyglycolic acid, polylactic acid, poly(lactic-co-glycolic) acid, poly(caprolactone).

27. The implantable matrix of claim 25, wherein the hydrogel is at least one of a naturally-derived hydrogel and a synthetic hydrogel.

28. The implantable matrix of claim 18, wherein the matrix comprises a hydrogel comprising thrombin and fibrinogen.

29. The implantable matrix of claim 28, wherein the thrombin is at a concentration of between about 0.5 units/mL and about 100 units/mL.

30. The implantable matrix of claim 28, wherein the fibrinogen is at a concentration of between about 3 units/mL and about 100 units/mL.

31. The implantable matrix of claim 18, wherein the matrix is implanted into at least one body region of a patient selected from the group consisting of: arms, legs, knees, hips, elbows, wrists, spine, stomach, blood vessels, and intestines.

32. A method of forming a medicament-producing implantable matrix, comprising:
combining at least one population of cells with a solubilized matrix;
casting the combination into a frame; and
polymerizing the solubilized matrix.

33. The implantable matrix of claim 32, wherein the at least one population of cells produces one or more molecules selected from the group consisting of: Ocrelizumab, Natalizumab, Pembrolizumab, Infliximab, Vedolizumab, Dabrafenib, Lecanemab, or interferon beta-1a.

34. The implantable matrix of claim 32, wherein the at least one population of cells produces one or more molecules selected from the group consisting of: Factor VII, Factor VIII, Factor IX, Factor X, Von Willebrand factor, Protein C, human albumin, human Immune Globulin, testosterone, human Htt, or P42.

35. The method of claim 32, wherein the at least one population of cells produces interleukin-10.

36. The method of claim 32, wherein the at least one population of cells produces one or more of a sirolimus or a tacrolimus.

37. The method of claim 32, wherein the at least one population of cells comprises mesenchymal stem cells.

38. The method of claim 32, wherein the at least one population of cells comprises major histocompatibility complex class I and II knock-out mesenchymal stem cells.

39. The method of claim 32, wherein the matrix comprises at least one selected from the group consisting of: a hydrogel and a biodegradable polymer.

40. The method of claim 39, wherein the biodegradable polymer is one selected from the group consisting of: polyglycolic acid, polylactic acid, poly(lactic-co-glycolic) acid, poly(caprolactone).

41. The method of claim 39, wherein the hydrogel is at least one of a naturally-derived hydrogel and a synthetic hydrogel.

42. The implantable matrix of claim 32, wherein the matrix comprises a Fas receptor activating molecule.

43. The implantable matrix of claim 42, wherein the Fas receptor activating molecule comprises a Fas ligand.

44. The method of claim 32, wherein the matrix comprises a hydrogel comprising thrombin and fibrinogen.

45. The method of claim 44, wherein the thrombin is at a concentration of between about 0.5 units/mL and about 100 units/mL.

46. The method of claim 44, wherein the fibrinogen is at a concentration of between about 3 units/mL and about 100 units/mL.

47. The method of claim 32, wherein the matrix is implanted into at least one body region of a patient selected from the group consisting of: knees, hips, elbows, wrists, spine, stomach, and intestines.

48. An apparatus, comprising:
a tubular support graft comprising a matrix and at least one population of cells, wherein the tubular support graft is configured to be concentrically positioned around a tubular vascular graft.

49. The apparatus of claim 48, wherein the at least one population of cells produces one or more molecules selected from the group consisting of: Ocrelizumab, Natalizumab, Pembrolizumab, Infliximab, Vedolizumab, Dabrafenib, Lecanemab, or interferon beta-1a.

50. The apparatus of claim 48, wherein the at least one population of cells produces one or more molecules selected from the group consisting of: Factor VII, Factor VIII, Factor IX, Factor X, Von Willebrand factor, Protein C, human albumin, human Immune Globulin, testosterone, human Htt, or P42.

51. The apparatus of claim 48, wherein the at least one population of cells produces interleukin-10.

52. The apparatus of claim 48, wherein the at least one population of cells produces one or more of a sirolimus or a tacrolimus.

53. The apparatus of claim 48, wherein the at least one population of cells comprises monocytes.

54. The apparatus of claim 48, wherein the at least one population of cells comprises mesenchymal stem cells.

55. The apparatus of claim 48, wherein the at least one population of cells comprises major histocompatibility complex class I and II knock-out mesenchymal stem cells.

56. The apparatus of claim 48, wherein the matrix comprises at least one selected from the group consisting of: a hydrogel and a biodegradable polymer.

57. The apparatus of claim 56, wherein the biodegradable polymer is one selected from the group consisting of: polyglycolic acid, polylactic acid, poly(lactic-co-glycolic) acid, poly(caprolactone).

58. The apparatus of claim 56, wherein the hydrogel is at least one of a naturally-derived hydrogel and a synthetic hydrogel.

59. The implantable matrix of claim 48, wherein the matrix comprises a Fas receptor activating molecule.

60. The implantable matrix of claim 48, wherein the Fas receptor activating molecule comprises a Fas ligand.

61. The apparatus of claim 48, wherein the matrix comprises a hydrogel comprising thrombin and fibrinogen.

62. The apparatus of claim 61, wherein the thrombin is at a concentration of between about 0.5 units/mL and about 100 units/mL.

63. The apparatus of claim 61, wherein the fibrinogen is at a concentration of between about 3 units/mL and about 100 units/mL.

64. The apparatus of claim 48, wherein the tubular support graft is formed by one of molding, casting, planar tissue culture, electrospinning, and 3-dimensional printing.

65. The apparatus of claim 48, wherein a length of the tubular support graft is less than a length of the tubular vascular graft.

66. The apparatus of claim 48, wherein a length of the tubular support graft is greater than a length of the tubular vascular graft.

67. The apparatus of claim 48, wherein a length of the tubular vascular graft is between 1 cm and 100 cm.

68. An apparatus, comprising:
a tubular support graft, wherein the tubular support graft is configured to be concentrically positioned around a vascular graft; and
a matrix comprising at least one population of cells and disposed on an external surface of the tubular support graft.

69. The apparatus of claim 68, wherein the at least one population of cells produces one or more molecules selected from the group consisting of: Ocrelizumab, Natalizumab, Pembrolizumab, Infliximab, Vedolizumab, Dabrafenib, Lecanemab, or interferon beta-1a

70. The apparatus of claim 68, wherein the at least one population of cells produces one or more molecules selected from the group consisting of: Factor VII, Factor VIII, Factor IX, Factor X, Von Willebrand factor, Protein C, human albumin, human Immune Globulin, testosterone, human Htt, or P42.

71. The apparatus of claim 68, wherein the at least one population of cells produces interleukin-10.

72. The apparatus of claim 68, wherein the at least one population of cells produces one or more of a sirolimus or a tacrolimus.

73. The apparatus of claim 68, wherein the at least one population of cells comprises mesenchymal stem cells.

74. The apparatus of claim 68, wherein the at least one population of cells comprises major histocompatibility complex class I and II knock-out mesenchymal stem cells.

75. The apparatus of claim 68, wherein the tubular support graft comprises one or more selected from the group consisting of: fibrin, collagen, alginate, gelatin, chitosan, dextran, hyaluronic acid, or PEG.

76. The apparatus of claim 68, wherein the tubular support graft comprises a combination of: PEG and chitosan, PEG and gelatin, PEG and hyaluronic acid, PAM and gelatin, or PVA and gelatin.

77. The apparatus of claim 68, wherein the matrix comprises at least one selected from the group consisting of: a hydrogel and a biodegradable polymer.

78. The apparatus of claim 77, wherein the biodegradable polymer is one selected from the group consisting of: polyglycolic acid, polylactic acid, poly(lactic-co-glycolic) acid, poly(caprolactone).

79. The apparatus of claim 77, wherein the hydrogel is at least one of a naturally-derived hydrogel and a synthetic hydrogel.

80. The apparatus of claim 68, wherein the matrix comprises a hydrogel comprising thrombin and fibrinogen.

81. The apparatus of claim 80, wherein the thrombin is at a concentration of between about 0.5 units/mL and about 100 units/mL.

82. The apparatus of claim 80, wherein the fibrinogen is at a concentration of between about 3 units/mL and about 100 units/mL.

83. The apparatus of claim 68, wherein the tubular support graft is formed by one of molding, casting, planar tissue culture, electrospinning, and 3-dimensional printing.

84. The apparatus of claim 68, wherein a length of the tubular support graft is less than a length of the tubular vascular graft.

85. The apparatus of claim 68, wherein a length of the tubular support graft is greater than a length of the tubular vascular graft.

86. The apparatus of claim 68, wherein a length of the vascular graft is between 1 cm and 100 cm.

87. A method for forming a functionalized tubular support graft for a vascular graft, comprising:
mounting a tubular support graft to a support mandrel;
rotating the support mandrel at a predetermined speed;
depositing, via a 3-dimensional printer, droplets of a mixture of hydrogel and at least one population of cells onto an external surface of the tubular support graft; and
after curing, removing the functionalized tubular support graft from the support mandrel.

88. The method of claim 87, wherein the at least one population of cells produces one or more molecules selected from the group consisting of: Ocrelizumab, Natalizumab, Pembrolizumab, Infliximab, Vedolizumab, Dabrafenib, Lecanemab, or interferon beta-1a.

89. The method of claim 87, wherein the at least one population of cells produces one or more molecules selected from the group consisting of: Factor VII, Factor VIII, Factor IX, Factor X, Von Willebrand factor, Protein C, human albumin, human Immune Globulin, testosterone, human Htt, or P42.

90. The method of claim 87, wherein the at least one population of cells produces interleukin-10.

91. The method of claim 87, wherein the at least one population of cells produces one or more of a sirolimus or a tacrolimus.

92. The method of claim 87, wherein the at least one population of cells comprises monocytes.

93. The method of claim 87, wherein the at least one population of cells comprises mesenchymal stem cells.

94. The method of claim 87, wherein the at least one population of cells comprises major histocompatibility complex class I and II knock-out mesenchymal stem cells.

95. The apparatus of claim 87, wherein the tubular support graft comprises one or more selected from the group consisting of: fibrin, collagen, alginate, gelatin, chitosan, dextran, hyaluronic acid, or PEG.

96. The apparatus of claim 87, wherein the tubular support graft comprises a combination of: PEG and chitosan, PEG and gelatin, PEG and hyaluronic acid, PAM and gelatin, or PVA and gelatin.

97. The method of claim 87, further comprising, implanting the vascular graft of the functionalized tubular support graft into an arm of a patient.

98. The method of claim 87, further comprising, implanting the vascular graft of the functionalized tubular support graft adjacent to an artery of a patient.

99. The method of claim 87, wherein the vascular graft can comprises one or more selected from the group consisting of: fibrin, collagen, alginate, gelatin, chitosan, dextran, hyaluronic acid, or PEG.

100. The method of claim 87, wherein the vascular graft can comprises a combination of: PEG and chitosan, PEG and gelatin, PEG and hyaluronic acid, PAM and gelatin, or PVA and gelatin.

101. The method of claim 87 further comprising placing the tubular support graft on an outer surface of the vascular graft.
